SEQUENCE LISTING (2 sequences)

SEQ ID NO: 1 - RNA, 735 nt, unidentified
  source 1..735: mol_type = other RNA
atgttcagcgtatttgaggaaatcacaagaattgtagttaaggagatggatgctggaggggatatgattgccgttagaagccttgttgatgctgatagattccgctgcttccatctggtgggggagaagagaactttctttggatgccggcactacacaacaggcctcaccctgatggacattctggacacagatggggacaagtggttagatgaactggattctgggctccaaggtcaaaaggctgagtttcaaattctggataatgtagactcaacgggagagttgatagtgagattacccaaagaaataacaatttcaggcagtttccagggcttccaccatcagaaaatcaagatatcggagaaccggatatcccagcagtatctggctacccttgaaaacaggaagctgaagagggaactacccttttcattccgatcaattaatacgagagaaaacctgtatctggtgacagaaactctggagacggtaaaggaggaaaccctgaaaagcgaccggcaatataaattttggagccagatctctcagggccatctcagctataaacacaagggccaaagggaagtgaccatccccccaaatcgggtcctgagctatcgagtaaagcagcttgtcttccccaacaaggagacgatgaatattcatttcaggggcaaaacaaaatcctttccagaagagaaggatggtgcttcatcctgtttaggaaagtaa

SEQ ID NO: 2 - protein, 244 aa, unidentified
MFSVFEEITRIVVKEMDAGGDMIAVRSLVDADRFRCFHLVGEKRTFFGCRHYTTGLTLMDILDTDGDKWLDELDSGLQGQKAEFQILDNVDSTGELIVRLPKEITISGSFQGFHHQKIKISENRISQQYLATLENRKLKRELPFSFRSINTRENLYLVTETLETVKEETLKSDRQYKFWSQISQGHLSYKHKGQREVTIPPNRVLSYRVKQLVFPNKETMNIHFRGKTKSFPEEKDGASSCLGK

(19) United States
(12) Patent Application Publication
(10) Pub. No.: US 2024/0415781 A1
(43) Pub. Date: Dec. 19, 2024

(54) PYROPTOSIS-TRIGGERING LIPID NANOPARTICLE

(71) Applicant: NEW JERSEY INSTITUTE OF TECHNOLOGY, Newark, NJ (US)

(72) Inventors: Xiaoyang Xu, Livingston, NJ (US); William Ho, Newark, NJ (US); Fengqiao Li, Harrison, NJ (US)

(73) Assignee: NEW JERSEY INSTITUTE OF TECHNOLOGY, Newark, NJ (US)

(21) Appl. No.: 18/742,581

(22) Filed: Jun. 13, 2024

Related U.S. Application Data
(60) Provisional application No. 63/472,681, filed on Jun. 13, 2023.

(51) Int. Cl. A61K 9/51; A61K 38/00; A61P 35/00; C07K 14/47; C12N 15/88

(52) U.S. Cl. CPC A61K 9/5123; A61P 35/00; C07K 14/47; C12N 15/88; A61K 38/00

(57) ABSTRACT

A lipid nanoparticle including: an ionizable cationic lipid compound including a reaction product of an amino alcohol and one or more lipid acids having from 4 to 26 carbons; one or more lipid components selected from a helper neutral lipid, a PEG-modified lipid, a phospholipid, and cholesterol; and a nucleic acid agent, the nucleic acid agent encoding an N-terminal domain of gasdermin. A lipid nanoparticle including: an ionizable cationic lipid compound having a structure of Formula (I); one or more lipid components selected from a helper neutral lipid, a PEG-modified lipid, a phospholipid, and cholesterol; and a single-agent mRNA, the single-agent mRNA encoding an N-terminal domain of gasdermin and including a polynucleotide sequence having at least 90% sequence identity to SEQ ID NO. 1. A method of treating a tumor, the method comprising administering a pharmaceutical composition to a subject in need thereof, the pharmaceutical composition comprising the lipid nanoparticle.

### CROSS-REFERENCE TO RELATED APPLICATIONS

This application claims priority to U.S. Provisional Application Ser. No. 63/472,681, filed Jun. 13, 2023, the entirety of which is hereby incorporated by reference herein.

### REFERENCE TO THE SEQUENCE LISTING

The Sequence Listing XML file submitted herewith, identified as “NJI0132-00US_sequence_listing.xml” (3,764 bytes, created Jun. 12, 2024), is hereby incorporated by reference herein.

### TECHNICAL FIELD

The present disclosure relates to gene delivery and therapy, such as pyroptosis-triggering mRNA lipid nanoparticles and their use in treating immunotherapy-resistant tumors.

### BACKGROUND

Checkpoints are a type of drug that blocks proteins. These are made by some immune system cells, such as T cells. These checkpoints serve to control immune responses and prevent certain immune system cells from killing tumor cells. When these checkpoints are blocked, the killing of tumor cells by certain immune system cells is enhanced.

Cancer immunotherapy, especially immune checkpoint blockade (ICB) therapy, is a major therapeutic modality and has prolonged the overall survival in many cancers¹⁻⁴. However, only a minority of patients experience a complete response to immunotherapy (10-30% in solid tumors)⁵⁻⁷, in part because of the highly immunosuppressive tumor microenvironment (TME) in immunologically “cold” tumors⁸⁻¹⁰, which are tumors that have a limited or no underlying immune response. Synergistically eliciting T-cell immune responses with inflammatory cytokines or immune agonists in cancer immunotherapy is a promising strategy to relieve immunosuppression and activate T cells¹¹⁻¹³. However, effective antitumor immunity requires activating all the steps of the cancer-immunity cycle, including immunogenic cell death (ICD), maturation of antigen-presenting cells, such as dendritic cells (DCs), priming and activation of T cells, recruitment of tumor-infiltrating immune cells, and production of inflammatory cytokines¹⁴⁻¹⁷. Unfortunately, even combinational therapies using multiple agents often lead to failure in the inhibition of cancer cells.

There is thus an unmet need for new therapeutic strategies to induce efficient antitumor immunity and broaden the scope of immunotherapy.

### SUMMARY

In accordance with embodiments of the present disclosure, an mRNA lipid nanoparticle-mediated pyroptosis process is disclosed that could sensitize immunologically “cold” tumors to checkpoint immunotherapy. Disclosed in some embodiments is a single-agent mRNA-based pyroptosis nanomedicine method that initiates the cancer-immunity cycle and converts “cold” tumors into inflammatory cytokine-expressing and T cell-infiltrated “hot” tumors to effectively treat immunologically “cold” tumors. A lipid nanoparticle including an ionizable cationic lipid compound comprising a reaction product of an amino alcohol and one or more lipid acids having from 4 to 26 carbons; one or more other lipid components selected from a helper neutral lipid, a PEG-modified lipid, a phospholipid, cholesterol, and combinations thereof; and a nucleic acid agent encoding an N-terminal domain of gasdermin. A lipid nanoparticle including an ionizable cationic lipid compound of Formula (I); one or more other lipid components selected from a helper neutral lipid, a PEG-modified lipid, a phospholipid, cholesterol, and combinations thereof; and a single-agent mRNA encoding an N-terminal domain of gasdermin and comprising a polynucleotide sequence having at least 90% sequence identity to SEQ ID NO. 1. A method of treating a tumor, the method comprising administering a pharmaceutical composition to a subject in need thereof, the pharmaceutical composition comprising the lipid nanoparticle.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a schematic illustration of intratumoral administration of mRNA lipid nanoparticles (LNPs) according to some embodiments;

FIGS. 2A-2E show experimental results of lipid nanoparticles delivering mRNA encoding the N-terminal domain of GSDMB into cells to induce pyroptosis, according to some embodiments;

FIGS. 3A-3C show experimental results of GSDMB^{NT} mRNA@LNPs promoting immunogenic cell death (ICD) in vitro according to some embodiments;

FIGS. 4A-4E show experimental results of treatment with GSDMB^{NT} mRNA@LNPs promoting tumor control in an anti-PD-1-resistant 4T1 breast cancer mouse model according to some embodiments;

FIGS. 5A-5E show experimental results of treatment with GSDMB^{NT} mRNA@LNPs inducing ICD in an anti-PD-1-resistant 4T1 breast cancer mouse model according to some embodiments;

FIGS. 6A-6H show experimental results of treatment with GSDMB^{NT} mRNA@LNPs enhancing potent antitumor activity in an aggressive melanoma mouse model according to some embodiments;

FIGS. 7A-7D show experimental results of treatment with GSDMB^{NT} mRNA@LNPs remodeling the tumor microenvironment in an aggressive melanoma mouse model according to some embodiments;

FIGS. 8A-8E shows experimental results of local combinational treatment of GSDMB^{NT} mRNA@LNPs and aPD-1 controling tumor burden at distant sites according to some embodiments;

FIG. 9A schematically illustrates a chemical synthesis route of AA3-DLin according to some embodiments;

FIG. 9B schematically illustrates an ionizable cationic lipid nanoparticle (LNP) and a therapeutic lipid nanoparticle encapsulating a nucleic acid agent according to some embodiments;

FIG. 10 schematically illustrates a combined AA3-DLin ionizable cationic lipid nanoparticle (LNP) and GSDM according to some embodiments;

FIGS. 11A-11B show experimental results of native agarose gel electrophoresis to identify the size of synthetic mRNAs according to some embodiments;

FIG. 12 shows experimental results of fluorescence imaging of HEK 293 and HeLa cells transfected by GFP mRNA-encapsulating AA3-DLin LNPs according to some embodiments;

FIG. 13 shows experimental results of representative images of HEK 293 and 4T1 cells transfected by mRNA/LNPs according to some embodiments;

FIG. 14 shows experimental results of cell morphologies of treated HEK293, HeLa, 4T1, and B16F10 cells according to some embodiments;

FIGS. 15A-15D show experimental results of percentages of apoptotic cells after transfection with GSDMB^{NT} mRNA@LNPs at various mRNA concentrations according to some embodiments;

FIGS. 16A-16B show experimental results of in vivo bioluminescence of luciferase mRNA-encapsulating LNPs of intratumoral injection in orthotopic 4T1 tumors according to some embodiments;

FIG. 17 shows experimental results of changes in body weight in 4T1-bearing mice and B16F10-bearing mice with varying treatments according to some embodiments;

FIGS. 18A-18B show experimental results of tumor section images of in vivo pyroptosis cells in the orthotopic 4T1-bearing tumor model and B16F10-bearing tumor model according to some embodiments;

FIG. 19A shows experimental results of tumor section images of in vivo pyroptosis cells in major organs in the combinational treatment of aPD-1 and GSDMB^{NT} mRNA@LNPs in B16F10-bearing mice according to some embodiments;

FIG. 19B shows experimental results of H&E staining of major organs in the combinational treatment of aPD-1 and GSDMB^{NT} mRNA@LNPs in B16F10-bearing mice according to some embodiments; and

FIGS. 20A-20E show experimental results of primary gating strategies for flow cytometric analysis of cells according to some embodiments.

### DETAILED DESCRIPTION

Before describing several exemplary embodiments of the invention, it is to be understood that the invention is not limited to the details of construction or process steps set forth in the following description. The invention is capable of other embodiments and of being practiced or being carried out in various ways.

Any combination and/or permutation of the embodiments is envisioned. Other objects and features will become apparent from the following detailed description considered in conjunction with the accompanying drawings. It is to be understood, however, that the drawings are designed as an illustration only and not as a definition of the limits of the present disclosure.

To fully understand the advantages of the present disclosure, a brief overview of pyroptosis is discussed below. Pyroptosis is a type of inflammatory programmed cell death that is triggered by the proteolytic cleavage of gasdermin (GSDM) family proteins 18. The GSDMs are normally self-inhibited through the intramolecular interaction of their N-terminal and C-terminal domains. Upon cleavage by specific caspases and other proteases in the linker region, the necrotic N-terminal domain form oligomers and translocate to the plasma membrane. The free N-terminal domain binds to lipid components and forms pores in the cell membrane, resulting in rapid plasma membrane rupture and release of danger-associated molecular patterns (DAMPs) and proinflammatory cytokines 19-22. Immune cells recognize certain DAMPs and then trigger a series of immune responses, including the activation and infiltration of immune cells 23.24. Additionally, released proinflammatory cytokines through the pyroptotic pore contribute to reversing the immunosuppressive tumor microenvironment (TME). Although these are encouraging discoveries, low GSDM expression in many cancers 19.20 and the complex cleavage process prevent delivering proteases to trigger pyroptosis for antitumor immunity.

Some embodiments provide methods that induce pyroptosis by direct delivery of the N-terminal GSDM domain to elicit a series of events in the cancer-immunity cycle. The method could transform “cold” tumors into “hot” tumors.

The present inventors recently developed a novel formulation to synthesize ionizable cationic lipid nanoparticles (LNPs), termed AA3-Dlin LNPs, with superior safety and outstanding mRNA translation efficiency in vitro and in vivo 25. In some embodiments, mRNA/LNPs are synergistically combined with GSDM for cancer treatment by triggering pyroptosis. In some embodiments, a MRNA lipid nanoparticle is disclosed that could encode only the N-terminus of gasdermin to trigger pyroptosis.

Some embodiments are directed to an amino alcohol mediated ionizable cationic lipid nanoparticles (AA-LNPs) platform. It should be understood that embodiments can generally be applied to other nucleic acid agents or other lipid nanoparticles situated within or encapsulated within other LNPs.

In some embodiments, an mRNA-based nanomedicine process is disclosed wherein the AA3-Dlin LNP formulation could encapsulate a single-agent mRNA encoding the GSDMB N-terminal domain, termed GSDMB^{NT} mRNA@LNPs. The GSDMB^{NT} mRNA@LNP formulation could be self-assembled by an ionizable cationic lipid (AA3-Dlin), phospholipid (for example, DOPE), cholesterol, and/or PEG. Without being bound by theory, it is believed that GSDMB^{NT} mRNA could be encapsulated inside the LNPs via electrostatic interactions.

In some embodiments, the AA3-Dlin LNP formulation encapsulates a nucleic acid agents, such as a DNA, a siRNA, a microRNA, an mRNA, a RNAi, a plasmid, or their antisense, single-stranded, double-stranded, or circular varieties, encoding the GSDMB N-terminal domain. In some embodiments, the N-terminal domain is selected from one or more of gasdermin-A (GSDM-A), gasdermin-B (GSDM-B), gasdermin-C(GSDM-C), gasdermin-D (GSDM-D), and gasdermin-E (GSDM-E).

In some embodiments, the N-terminal domain is an N-terminal domain of GSDM-B and the nucleic acid agent has a polynucleotide sequence given by SEQ ID NO. 1. In some embodiments, the polynucleotide sequence has 100% sequence identity to SEQ ID NO. 1. In some embodiments, the polynucleotide sequence has at least about 99.5% sequence identity to SEQ ID NO. 1. In some embodiments, the polynucleotide sequence has at least about 99% sequence identity to SEQ ID NO. 1. In some embodiments, the nucleic acid agent has at least about 98% sequence identity to SEQ ID NO. 1. In some embodiments, polynucleotide sequence has at least about 95% sequence identity to SEQ ID NO. 1. In some embodiments, polynucleotide sequence has at least about 90% sequence identity to SEQ ID NO. 1. In some embodiments, the polynucleotide sequence has at least about 80% sequence identity to SEQ ID NO. 1. In some embodiments, the polynucleotide sequence has at least about 70% sequence identity to SEQ ID NO. 1.

In some embodiments, the N-terminal domain is an N-terminal domain of GSDM-B and the polynucleotide encodes a polypeptide sequence given by SEQ ID NO. 2. In some embodiments, the N-terminal domain has 100% sequence identity to SEQ ID NO. 2. In some embodiments, the N-terminal domain has at least about 99.5% sequence identity to SEQ ID NO. 2. In some embodiments, the N-terminal domain has at least about 99% sequence identity to SEQ ID NO. 2. In some embodiments, the N-terminal domain has at least about 98% sequence identity to SEQ ID NO. 2. In some embodiments, the N-terminal domain has at least about 95% sequence identity to SEQ ID NO. 2. In some embodiments, the N-terminal domain has at least about 90% sequence identity to SEQ ID NO. 2. In some embodiments, the N-terminal domain has at least about 80% sequence identity to SEQ ID NO. 2. In some embodiments, the N-terminal domain has at least about 70% sequence identity to SEQ ID NO. 2.

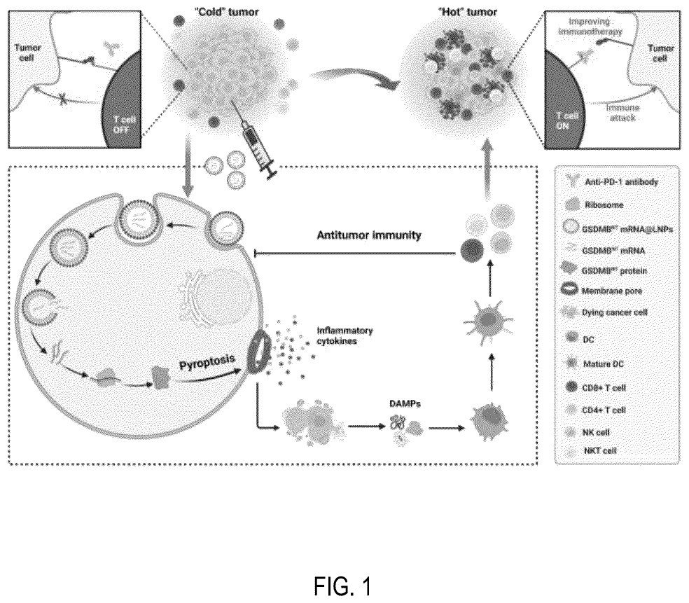

In some embodiments, the formulation is delivered into tumor tissue where the mRNA is translated into the N-terminus of the GSDMB protein, triggering pyroptosis directly without protease cleavage. Pyroptosis can initiate the expression of proinflammatory cytokines, induce ICD, as well as activate and recruit immune cells in tumor sites, which forms a positive feedback loop in antitumor immunity. This cancer-immunity cycle can create a favorable immunogenic “hot” tumor microenvironment that can further sensitize tumors to ICB-mediated immunotherapy, such as anti-PD-1 antibody, as shown in FIG. 1.

FIG. 1 shows that intratumoral administration of mRNA lipid nanoparticles encoding only the N-terminal domain of GSDMB can trigger pyroptosis, elicit antitumor immunity, and/or facilitate anti-PD-1-mediated immunotherapy in immunologically “cold” tumors. As illustrated in FIG. 1, in some embodiments, mRNA lipid nanoparticles could be administered to a cold tumor, thereby triggering pyroptosis, which could convert the “cold” tumor to a “hot” tumor.

As shown in FIG. 1, the process of cellular internalization of LNPs starts with endocytosis, followed by escape from the endosome, degradation of LNPs, and release of mRNA into the cytosol. Subsequently, the GSDMB^{NT} mRNA is translated into GSDMB^{NT} proteins to induce pyroptosis directly, without the involvement of protease cleavage, in a localized manner. Pyroptotic cell death further initiates the release of proinflammatory cytokines, induces the expression of DAMPs (non-limiting examples including ATP, HMGB1, CRT), activates the maturation of dendritic cells and recruits immune cells in the tumor microenvironment. Activated T cells directly induce the killing of tumor cells.

As described in further detail below, in vitro results of the present disclosure demonstrate that even low levels of tumor cell pyroptosis triggered by single-agent GSDMB^{NT} mRNA@LNPs can induce robust ICD. In multiple immunologically “cold” tumor models, the results of the present invention show that pyroptosis-triggering mRNA/LNPs can significantly inhibit tumor growth and extend overall survival, accompanied by stimulation of proinflammatory cytokines and promotion of the recruitment of immune cells in the TME. Moreover, pyroptosis-triggering mRNA/LNPs can significantly improve the therapeutic benefits of immune checkpoint inhibitor (anti-programmed death-1 antibody, aPD-1)-mediated immunotherapy and even achieve tumor elimination and long-term survival in both orthotopic 4T1 breast carcinoma and highly aggressive B16F10 melanoma models.

In addition, pyroptosis-triggering mRNA/LNPs can potently synergize with aPD-1-mediated immunotherapy, induce a local immune response and subsequently provoke a systemic effect, to eradicate large melanomas and inhibit the growth of distant tumors in a B16F10 dual-tumor model. Collectively, the single-agent pyroptosis-triggering mRNA/LNPs approach disclosed herein provides a facile and highly efficacious strategy to achieve potent antitumor immunity and enhance immunotherapy in immunologically “cold” tumors. In some embodiments, the methods disclosed herein provide a versatile platform that can be extended to other immunotherapies besides aPD-1, providing significant benefits for translational medicine.

Cancer immunotherapy is a major therapeutic modality for the treatment of many cancers. Thus, some embodiments of the disclosure activate or boost the ability of endogenous T cells within the tumor to recognize and destroy cancer cells through natural immune mechanisms 1-3,30. Particularly, the advent of ICB therapy has greatly advanced the field of cancer immunotherapy, as it has successfully been shown to inhibit checkpoint proteins to augment the host's immunologic activity against tumors 4. However, resistance to immunotherapy occurs frequently due to immunosuppressive cytokines and insufficient tumor-infiltrating immune cells in the TME, driving researchers to explore approaches to sensitize immunologically “cold” tumors.

Some embodiments of the present disclosure provide a general mRNA nanomedicine approach showing that single-agent mRNA/LNPs encoding GSDM N-terminal domain trigger inflammatory pyroptosis to turn “cold” tumors “hot”. The single-agent pyroptosis-triggering mRNA/LNPs enable robust antitumor immunity and reinforce aPD-1-mediated immunotherapy through reprograming the TME from immunosuppressive into immunostimulatory phenotype.

GSDM-mediated pyroptosis, a form of cell death accompanied by the secretion of multiple inflammatory cytokines, has recently attracted considerable attention as a unique mechanism in cancer immunotherapy 19-21. Evidence suggests that the expression of GSDM proteins is suppressed in many cancers 19.31. For example, GSDMB appears to be silenced in gastric and esophageal cancers 20. Thus, exogenous GSDMB may potentially act as a tumor suppressor by activating pyroptosis to achieve antitumor immunity. A benefit of the present invention is that single-agent GSDM-mediated pyroptosis not only induces cell death directly but also activates multiple pathways of immunity to create a self-sustaining cancer-immunity cycle, which is challenging in inflammatory cytokine or immune agonist therapies.

Moreover, the present disclosure demonstrates that even 20% cellular pyroptosis triggered by single-agent GSDMBA^{NT} mRNA@LNPs was adequate to promote robust immunogenic cell death, characterized by morphological changes, plasma membrane rupture, cell death, and DAMP secretion. In the 4T1 and B16F10 mouse tumor models, the results of the present invention further suggest that low levels of cancer cell pyroptosis activated the secretion of proinflammatory cytokines (such as TNF-α and IFN-γ), upregulated the expression of DAMPs, recruited the infiltration of various immune cells, and inhibited the growth of established tumors, thereby establishing a positive feedback loop to promote antitumor immunity. A recent study has shown that pyroptosis of less than 15% of tumor cells was sufficient to control over 4T1 mammary tumor graft 21. In comparison with other strategies in cancer immunotherapy that induce ICD, such as chemotherapy or thermotherapy, GSDM-mediated pyroptosis does not require the killing of large numbers of tumor cells to induce a robust immune response. In a clinical setting, this may be highly advantageous, due to reduced high-dose toxicity and avoiding side effects on normal tissues.

However, conventional GSDMB-mediated pyroptosis requires the caspase or granzyme A-dependent cleavage to free GSDB^{NT} in GSDMB-expressing cells²⁰. In some embodiments, utilizing GSDMB^{NT}-encoded mRNA/LNPs, free GSDB^{NT} was delivered into tumor tissue directly and trigger pyroptosis in a facile and highly efficient manner. Indeed, the results demonstrate that only GSDMB^{NT} mRNA@LNPs, but not mRNA@LNPs encoding the full length or C-terminal of GSDMB, induced extensive pyroptosis in transfected cells. When compared with the conventional protein delivery strategy to cleave full-length GSDM proteins, the process of the present invention shows that the direct delivery of N-terminus GSDM mRNA/LNPs could trigger pyroptosis simply, immediately, and effectively, which is encouraging for its clinical translation.

T-cell recruitment is a critical challenge for immunotherapy in “cold” tumors. The GSDMB^{NT} mRNA@LNP treatment of the present invention recruited tumor-infiltrating CD4+ and CD8⁺ T cells, probably because the treatment initiates or reinitiates a self-sustaining cycle of cancer immunity after intratumoral TNF-α and IFN-γ induction^{32,33}. The present inventors also discovered higher expression of ICD biomarkers in mice treated with pyroptosis-triggering mRNA/LNPs, especially in mice with the combinatorial treatment of GSDMB^{NT} mRNA@LNPs and aPD-1. These results reveal that pyroptosis-triggering mRNA/LNPs remodeled the immunosuppressive TME that promotes response to aPD-1. As expected, GSDMB^{NT} mRNA@LNPs improved the therapeutic benefits of aPD-1 immunotherapy, including prolonged survival, and significant inhibition of tumor growth. In addition, the present inventors discovered that a local immune response stimulated by GSDMB^{NT} mRNA@LNPs treatment in one lesion provoked a systemic antitumor response, contributing to control over distant untreated lesions. These results highlight the possibility to potentiate ICB-mediated immunotherapy in the clinic by synergy with N-terminus GSDM-mediated pyroptosis.

For clinical translation, one additional advantage of the pyroptosis-triggering mRNA/LNP approach of the present invention is the mRNA/LNP delivery system. mRNA nanomedicine-based gene therapy is a promising therapeutic modality for the treatment of various diseases, due to its excellent safety, quick manufacturing and production, and ability to encode proteins or gene-editing components such as cas9 protein³⁴⁻³⁷. Recently, the COVID-19 mRNA vaccines produced by Moderna and Pfizer-BioNTech highlight the potential of mRNA/LNP technology³⁸⁻⁴¹. Although these vaccines utilize mRNA therapeutics, as of now no mRNA nanomedicines have been approved for cancer treatment in clinics^{42,43}.

The synergistic combination of mRNA encoding inflammatory cytokines or immune agonists with immunotherapy has been extensively explored to enhance cancer immunotherapy. However, even mRNAs encoding multiple cytokines still failed to induce successful antitumor immunity. For example, the mRNA mixture of four cytokines, including interleukin-12 (IL-12) single chain, interferon-α (IFN-α), granulocyte-macrophage colony-stimulating factor, and IL-15 sushi, was able to induce a proinflammatory TME and boost antitumor T cell activity, while any single cytokine treatment failed to drive effective growth inhibition of established immunologically “cold” mouse tumors³⁴. The synergistic combination of pyroptosis-triggering mRNA/LNPs with immunotherapy provides new insights into developing single-agent mRNA nanomedicine for cancer treatment in future clinical practice.

In some embodiments, a unique single-agent mRNA nanomedicine that takes advantage of the newly discovered GSDM-mediated pyroptosis pathway and LNP-meditated mRNA delivery system to improve cancer immunotherapy is disclosed. The approach possesses many beneficial properties, such as: 1) GSDMB^{NT} mRNA@LNPs not only kill cancer cells directly but also elicit a robust and safe antitumor immunity using a single-agent mRNA; 2) GSDMB^{NT} mRNA@LNPs deliver the N-terminus of gasdermin to trigger rapid and efficacious pyroptosis without the need for protease cleavage; 3) GSDMB^{NT} mRNA@LNPs reverse the immunosuppressive TME and recruit tumor-infiltrating immune cells, turning “cold” tumors “hot”; 4) GSDMB^{NT} mRNA@LNPs could be synergized with immune checkpoint blockades to strengthen immunotherapy efficacy, resulting in long-term overall survival, elimination of treated tumors, and stabilization of distant lesions; 5) GSDMB^{NT} mRNA@LNPs sensitize aPD-1-mediated immunotherapy in a general manner, thereby they may serve as a basic universal platform to potentially enhance other immunotherapy modalities, such as T cell-based therapies and cancer vaccines. Overall, the single-agent pyroptosis-triggering mRNA nanomedicine therapy of the present invention is simple and highly efficacious, holding a great potential for clinical translation.

The materials and the methods of the present disclosure used in some embodiments will be described below. While the embodiment discusses the use of specific compounds and materials, it is understood that the present disclosure could employ other suitable compounds or materials. Similar quantities or measurements may be substituted without altering the method embodied below.

### EXAMPLES

### Example 1: Design, Synthesis and Screening of an Ionizable Cationic Lipid Library

Preparation and Characterization of mRNA-Encapsulating LNPs

The sequences for in vitro transcription of mRNA, including T7 promoter, 5′ UTR, coding sequence, 3′ UTR, and poly(A) were cloned into pVAX1 vector using NEBuilder® HiFi DNA Assembly Cloning Kit. The mixture was then transformed into DH5a Competent Cells (ThermoFisher) by chemical transformation, and the synthesized plasmid was confirmed by Sanger Sequencing. Then, a linearized DNA template, including T7 promoter, 5′ UTR, coding sequence, 3′ UTR, and poly(A) was achieved by Bsal digestion. The DNA templates were purified by QIAquick Gel Extraction Kit (Qiagen) and confirmed by 1% agarose gel electrophoresis. All mRNAs were synthesized by in vitro transcription with ARCA (TriLink) or CleanCap (TriLink) and 100% pseudouridine-5′-triphosphate (APExBIO) using AmpliScribe T7-Flash Transcription Kit (Lucigen) following the manufacturer's instruction. Subsequently, mRNA was purified by RNA Clean & Concentrator (Zymo). The synthesized mRNAs were examined by 1% agarose gel electrophoresis and stored at −80° C. for future use.

LNP formulations were prepared as previously described 25. Briefly, lipids were dissolved in ethanol at molar ratios of 40:40:25:0.5 (AA3-DLin: DOPE: cholesterol: PEG-2000). The lipid mixture was combined with a 25 mM Sodium acetate buffer solution (pH 5.5) containing mRNA at a ratio of 20:1 (AA3-DLin: mRNA, wt./wt.) for in vitro study and 10:1 for in vivo study. Formulations were dialyzed against PBS (pH 7.4) in dialysis cassettes overnight.

The hydrodynamic diameters and zeta potentials of mRNA-encapsulating LNPs were analyzed by dynamic light scattering (DLS) on a Malvern Instruments Zetasizer HS III (Malvern, UK) at room temperature. Transmission electron microscopy (JEM-F200 TEM, USA) was performed to detect the morphology of mRNA-encapsulating LNPs. Furthermore, to investigate the stability, the size changes of mRNA-encapsulating LNPs in PBS, medium or medium with 10% FBS were measured at 0, 2, 4, 6, 8, 24, 48, and 72 hours.

### Design of an Ionizable Cationic Lipid Library

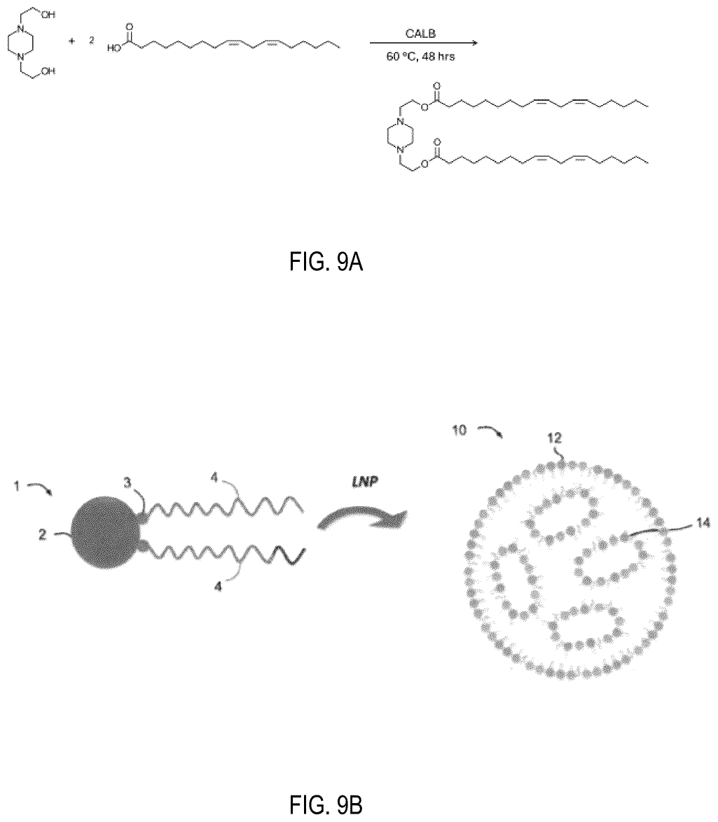

In some embodiments, an ionizable cationic lipid is generally composed of three parts: (1) one or more hydrophilic headgroups containing one or multiple ionizable amines for condensing negatively charged mRNA; (2) one or more hydrophobic hydrocarbon chains capable of promoting self-assembly and phospholipid membrane fusion; and (3) one or more degradable ester linkers connecting the headgroups with the hydrocarbon chains to potentially lower systemic cytotoxicity, as shown in FIG. 9B. In some embodiments, a new library of ionizable cationic lipids is provided, with ionizable amine headgroups derived from different amino alcohols and hydrocarbon lipid chains derived from commercially available lipid acids. In some embodiments, using CALB enzyme-assisted reaction, the hydroxyl groups were reacted with carboxylic acids via one-step high-efficiency esterification, as shown in FIG. 9A. An 18*8 library of lipid-like materials was synthesized by varying amino alcohols and lipid acids. These lipid-like materials with DOPE, cholesterol, and DMG-PEG were fabricated at a molar ratio of 50:10:38.5:1.5, which was a widely used formulation to form LNPs and delivered luciferase encoded mRNA (mLuc) in vitro to generate a luciferase expression heat map of lipid-like materials.

The top-performing ionizable cationic lipid was screened and termed as AA3-DLin, which is chemically composed of 1,4-Bis(2-hydroxyethyl) piperazine amine headgroups connected with two linoleic lipids by ester linkers as shown in the structure (FIGS. 9A-B). The AA3-DLin has the structure given by Formula (I):

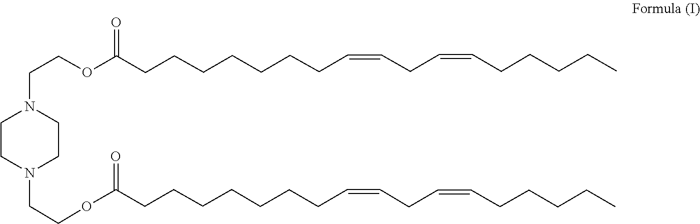 [SMILES: CCCCC/C=C\C/C=C\CCCCCCCC(=O)OCCN1CCN(CCOC(=O)CCCCCCC/C=C\C/C=C\CCCCC)CC1]

The AA3-DLin lipids were synthesized by high-efficiency CALB-mediated catalytic esterification and characterized by electrospray ionization (ESI) mass spectrometry, which showed a strong, clear and single peak denoting AA3-DLin with molecular weight (MW) of 699 and up to 96% purity. Nuclear magnetic resonance (NMR) spectroscopy and Fourier-transform infrared spectroscopy (FTIR) confirmed the chemical structure of AA3-DLin accordingly. In one or more embodiments, the ionizable cationic lipid molecules comprise a molecular weight in a range of from 200 to 2000 Daltons, including all values and subranges therebetween. In some embodiments, the ionizable cationic lipid molecules comprise a molecular weight in a range of from 200 to 400 Daltons, or from 400 to 600 Daltons, or from 600 to 800 Daltons, or from 800 to 1000 Daltons, or from 1000 to 1200 Daltons, or from 1200 to 1400 Daltons, or from 1400 to 1600 Daltons, or from 1600 to 1800 Daltons, or from 1800 to 2000 Daltons.

FIG. 9A shows a chemical synthesis route of AA3-DLin through CALB enzyme-assisted esterification according to one or more embodiments to prepare amino alcohol mediated ionizable cationic lipid compounds. In one or more embodiments, the reaction of amino alcohol and lipid acid is conducted at 60° C. for 48 hours in the presence of an enzyme, e.g., CALB. FIG. 9B illustrates a schematic view of an ionizable cationic lipid 1 comprising an amino alcohol mediated ionizable cationic lipid compound, according to one or more embodiments. An amine head 2 supplied by the amino alcohol is attached by an ester linker 3 to lipid chains 4. FIG. 9B also illustrates a schematic view of a therapeutic lipid nanoparticle 10. The therapeutic lipid nanoparticle 10 comprises an LNP fabricated by using one or more amino alcohol mediated ionizable cationic lipid compounds, with other lipid components, which assemble to form an outer shell 12 encapsulating a primary core, which includes therein a nucleic acid-based agent such as sgRNA. In some embodiments, the outer shell 12 could comprise an assembly of amino alcohol mediated ionizable cationic lipid compounds, with phospholipids, cholesterol and PEGylated lipids.

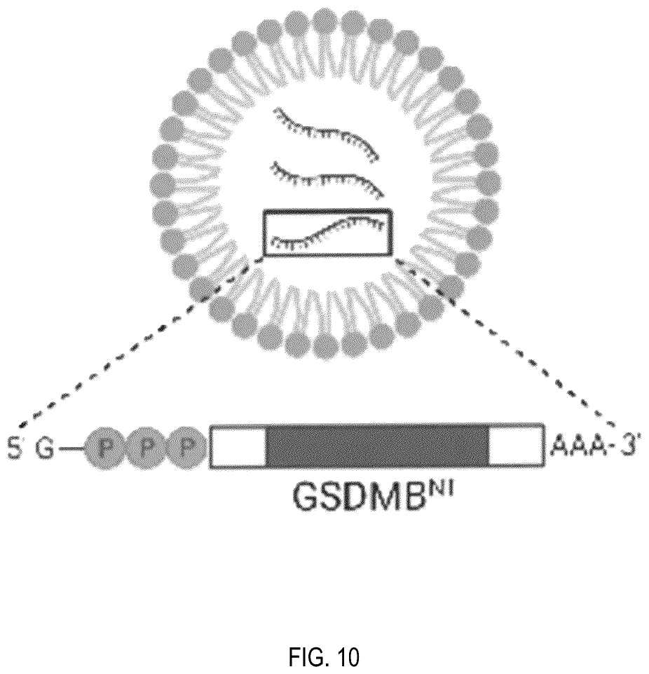

FIG. 10 provides a schematic view of AA3-DLin with GSDM N-terminal nucleic acid-based agents encapsulated within.

### Example 2: LNP Delivery of GSDMB^{NT} mRNA into Cells to Induce Pyroptosis

### Cell Culture and Transfection

HEK 293, Hela, 4T1, and B16F10-Luc cell lines were obtained from the American Type Culture Collection (ATCC). All cells were cultured in Dulbecco's modified Eagle's medium (DMEM) containing 10% FBS, 100 units/mL penicillin, and 100 mg/mL streptomycin. Cells were grown in a humidified atmosphere with 5% CO₂ at 37° C.

For in vitro transfection, 1 μg mRNA was encapsulated in LNPs as described herein. The formulated mixtures were added into a well of a 12-well plate containing 1.0 ml medium. At predetermined time points after transfection, the cells or supernatants were collected for further assays. For in vivo transfection, 10 μg mRNA was formulated as above and intratumorally administrated for various assays.

### In Vitro Pyroptosis Evaluation

To examine the changes in cell morphology after GSDMB^{NT} mRNA@LNPs transfection, HEK 293, Hela, 4T1, and B16F10-Luc cells were seeded in 35 mm Petri dishes containing 1.0 mL DMEM medium. After cell attachment, cells were treated with either GSDMB^{NT} mRNA, LNPs, or GSDMB^{NT} mRNA@LNPs. 24 hours after treatment, annexin V-FITC and propidium iodide were added to the cell culture medium before being subjected to imaging of living cells using a Nikon AIR+HD Confocal Microscope. The images shown herein are representative of at least three randomly selected fields. To quantitatively analyze pyroptotic cells, flow cytometry was performed to determine the number of annexin V-FITC and propidium iodide-positive cells. All cells collected from each 12-well plate were washed twice with Annexin V binding buffer and stained by using a FITC Annexin V Apoptosis detection kit with PI (BioLegend).

The cell viability was evaluated in a calcein—AM release assay. Briefly, 2% Triton X-100 was added to cells in the control group for 2 hours, which was used to determine the maximum release by nonspecific lysis. Cells without any treatment were used to determine the spontaneous release of calcein. Subsequently, 4 μg/mL calcein AM was added to the cell culture medium for 30 minutes at 37° C. The fluorescence intensity of the released calcein was measured under an excitation wavelength of 485 nm and an emission wavelength of 530 nm. The calcein release was calculated according to the formula: [(test release-spontaneous release)/(maximum release by nonspecific lysis-spontaneous release)].

### Cell Immunofluorescent Staining

HEK 293, Hela, 4T1, and B16F10-Luc cells were seeded on glass slides in 12-well plates and were incubated with either GSDMB^{NT} mRNA, LNPs, or GSDMB^{NT} mRNA@LNPs. After 48 hours, cells were washed twice with PBS and fixed with 4% paraformaldehyde for about 20 minutes at room temperature. Then, the cells were permeabilized with 0.1% Triton X-100 PBS (PBST) and blocked with a PBST blocking solution containing 5% goat serum for 1 hour at room temperature. Next, the cells were incubated in the diluted antibody in PBST in a humidified chamber overnight at 4° C. Subsequently, cells were washed with PBS three times and incubated in secondary antibody with Alexa Fluor 594 for 1 hour at room temperature. Then, cells were washed with PBS three times and stained with 4′,6-diamidino-2-phenylindole (DAPI) (Thermo Fisher Scientific) for 10 minutes to visualize cells nuclei. Finally, cells were sealed with a drop of mounting medium and analyzed by a Nikon AIR+HD Confocal Microscope at a wavelength of 594 nm. The images shown herein are representative of at least three randomly selected fields.

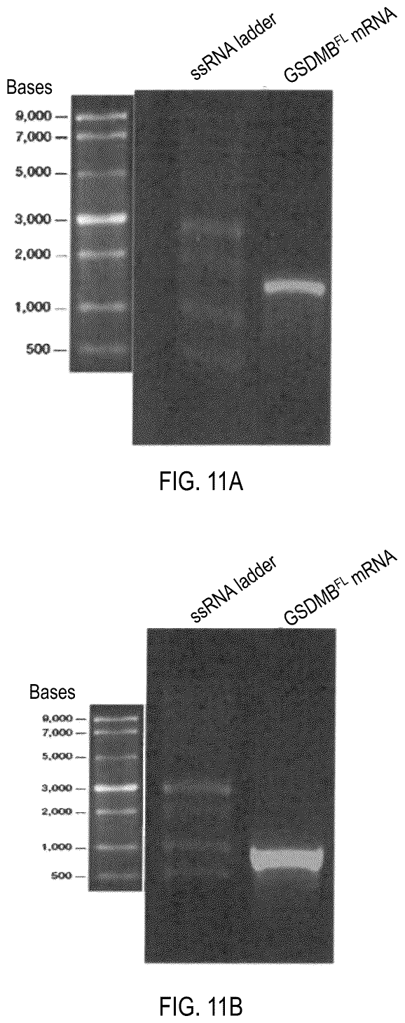

GSDMB^{NT} mRNA was first synthesized using an in vitro transcription method 26 with significant cap modification and sequence optimization. An agarose gel assay was performed to confirm the size of synthesized GSDMB^{NT} mRNA, as shown in FIG. 11. FIG. 11 depicts native agarose gel electrophoresis measurements that were used to identify the size of synthetic mRNAS encoding GSDMB full-length (GSDMBFL) (FIG. 11A) and GSDMB N-terminal (GSDMB^{NT}) (FIG. 11B).

Effective mRNA delivery relies on the physical and chemical characteristics of formulations. Therefore, in some embodiments, an AA3-Dlin LNP platform was then prepared to encapsulate GSDMB^{NT} mRNA (GSDMB^{NT} mRNA@LNPs), as previously reported 25. The morphology, hydrodynamic diameter, and zeta potential of GSDMB^{NT} mRNA@LNPs were evaluated by transmission electron microscopy (TEM) and dynamic light scattering (DLS), respectively.

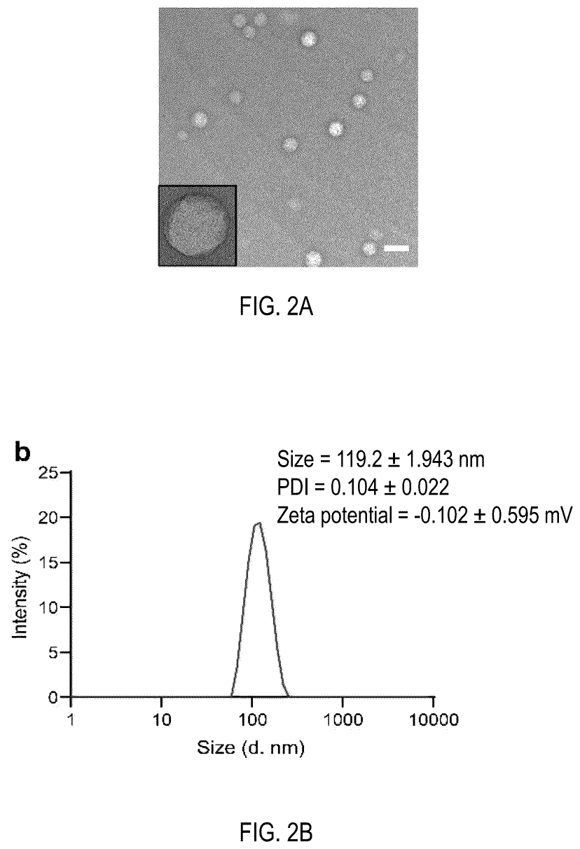

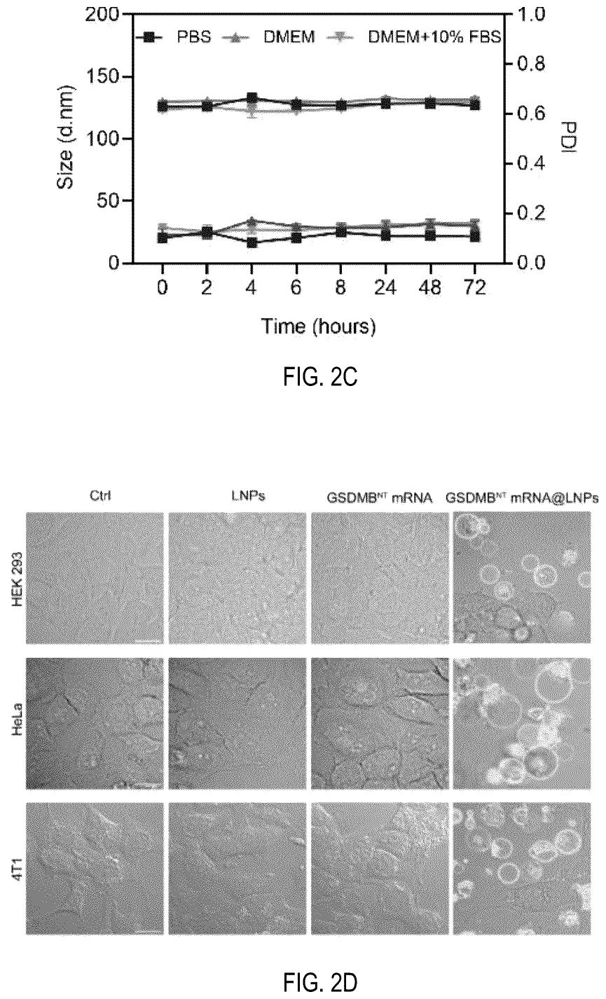

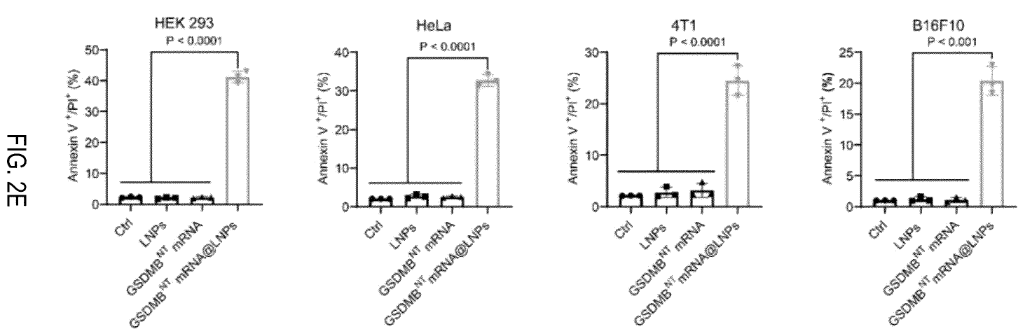

FIG. 2A provides results showing that the LNPs of some embodiments described herein can deliver mRNA encloding the N-terminal domain of GSDMB into cells and to induce pyroptosis in the cells. FIG. 2A shows a TEM image of GSDMB^{NT} mRNA@LNPs according to some embodiments, with the scale bar corresponding to 200 nm. FIG. 2B shows the particle size, polydispersity index (PDI), and zeta potential of GSDMB^{NT} mRNA@LNPs according to some embodiments as analyzed by DLS. FIG. 2C shows stability of GSDMB^{NT} mRNA@LNPs in PBS, DMEM, and DMEM containing 10% FBS. FIG. 2D shows cell morphologies of the treated HEK 293, HeLa, and 4T1 cells as detected using a confocal microscope, with scale bars corresponding to 20 μm. FIG. 2E shows the cell viability as measured by calcein release. FIG. 2F shows Annexin V-FITC and PI-positive apoptopic cells as determined by flow-cytometry analysis. All data are representative of either three or four independent experiments.

As shown in FIG. 2A, the TEM analysis revealed that GSDMB^{NT} mRNA@LNPs are spherical in morphology and have a smooth surface. The particle size, polydispersity index (PDI), and zeta potential of GSDMB^{NT} mRNA@LNPs as assessed from DLS was about 119.2±1.943 nm, 0.104±0.022 and −0.102±0.595, respectively, as shown in FIG. 2B. Importantly, no obvious changes were observed in particle size or PDI in phosphate-buffered saline (PBS) or cell culture medium within 72 hours, which revealed that GSDMB^{NT} mRNA@LNPs maintain stability in physiological environments, as shown in FIG. 2C.

Before the pyroptotic efficacy was evaluated, the transfection efficiency of AA3-Dlin LNPs encapsulating green fluorescent protein (GFP) mRNA as a reporter gene (termed GFP mRNA@LNPs) was tested in HEK 293 and HeLa cells.

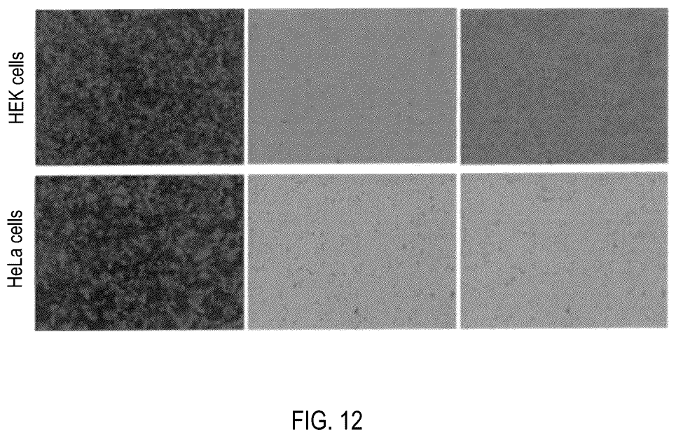

FIG. 12 shows fluorescence imaging of HEK 293 and HeLa cells transfected by GFP mRNA-encapsulating AA3-DLin LNPs after 24 hours of treatment, with the scale bar corresponding to 100 μm. As shown in FIG. 12, high expression of GFP was observed in transfected cells at 24 hours post-transfection.

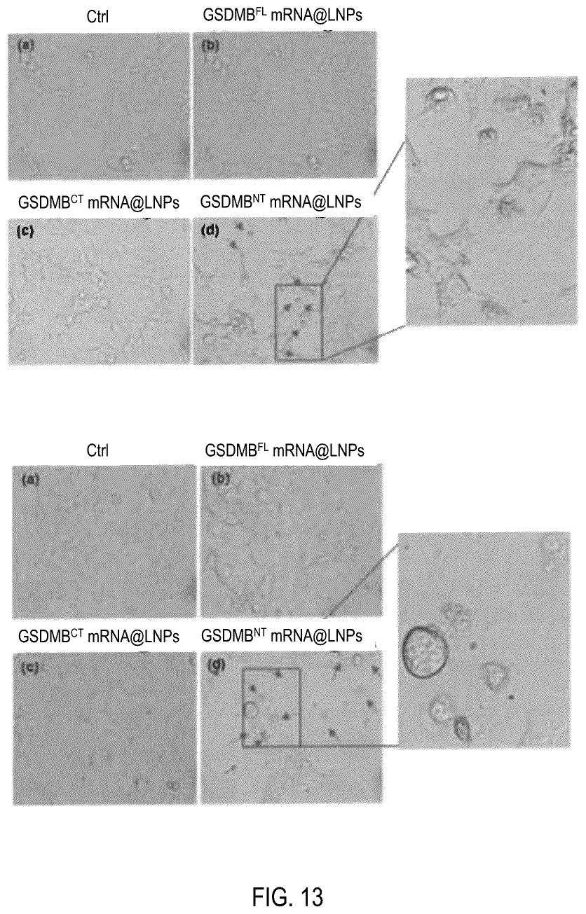

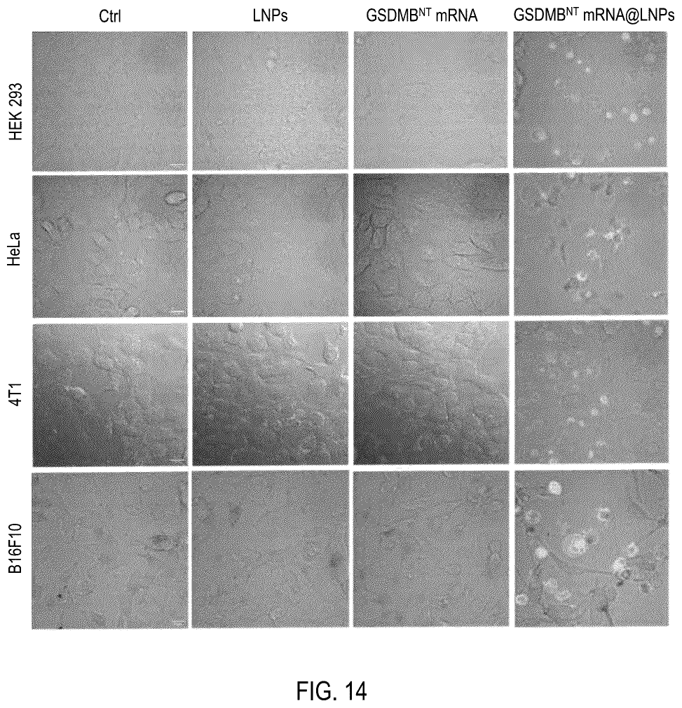

FIG. 13 shows representative images of HEK 293 (left) and 4T1 (right) cells transfected by mRNA/LNPs encoding GSDMB full-length (GSDMB^{FL}), GSDMB C-terminal (GSDMBCT), or GSDMB N-terminal (GSDMB^{NT}) after 24 hours of treatment. FIG. 14 shows cell morphologies of the treated HEK293, HeLa, 4T1, and B16F10 cells as detected using confocal microscopy. The scale bars correspond to 20 μm. Before imaging, cells were added with annexin V-FITC and propidium iodide (PI) for 15 minutes of incubation.

When treated with GSDMB^{NT} mRNA@LNPs, pyroptotic morphological changes involving cytoplasmic swelling and membrane rupture were observed in HEK 293, HeLa, 4T1, and B16F10 cells, as shown in FIGS. 2D and 13-14. These phenomena were not observed in the control groups including PBS and LNP-treated groups. Then, Annexin V/propidium iodide (PI) apoptosis assay was used to quantitate the lethal effect of GSDMB^{NT} mRNA@LNPs on the cancer cells.

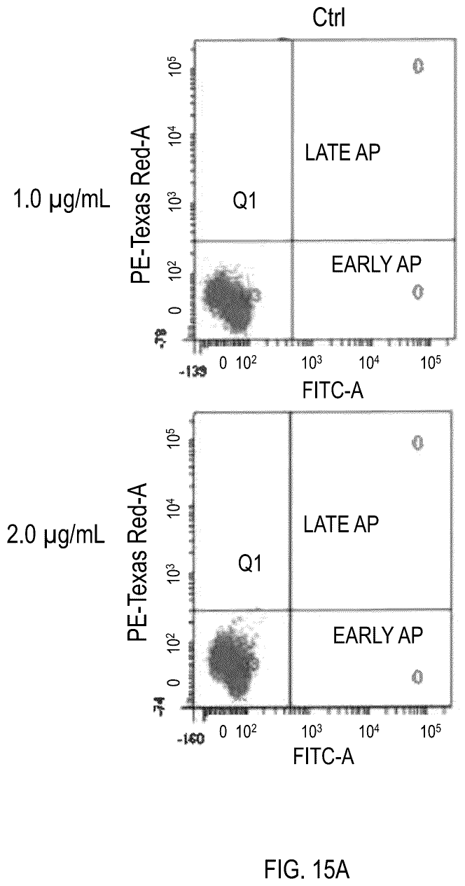

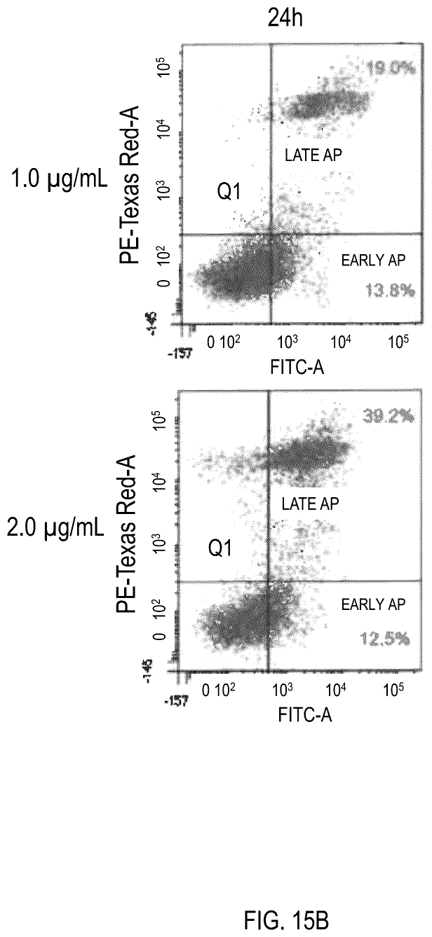

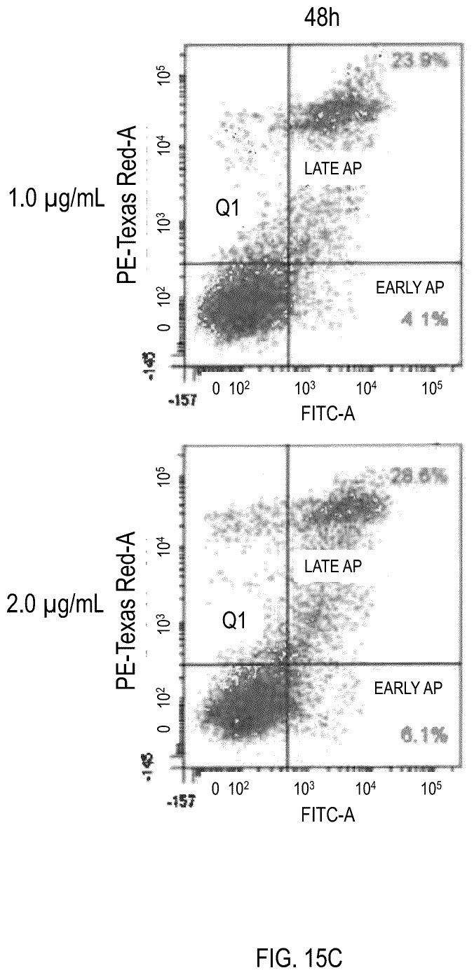

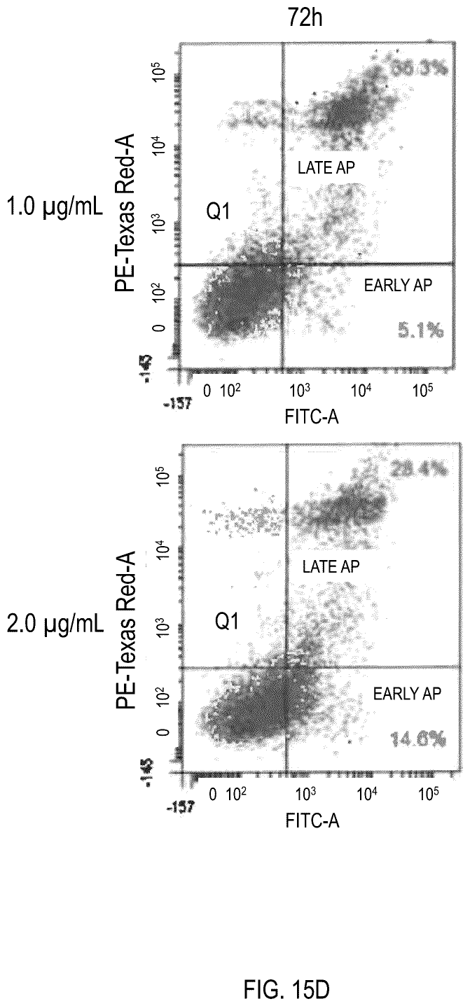

FIG. 15 shows the percentage of apoptopic cells as determined by staining with FITC-Annexin V/PI. HEK 293 cells were transfected with GSDMB^{NT} mRNA@LNPs at various mRNA concentrations for 24, 48, and 72 hours. FIG. 15 shows concentration-dependent and time-dependent pyroptosis of Hek 293 cells treated with GSDMB^{NT} mRNA@LNPs. About 40% of Hek 293 cells underwent pyroptosis after 24 hours of treatment with 1.0 μg/mL GSDMB^{NT} mRNA@LNPs in some embodiments, which indicates that a timeframe of 24 hours and dosage of 1.0 μg/mL of treatment is sufficient to trigger pyroptosis. It will be understood that the timeframe and the dosage could vary.

Under the same treatment time and dose conditions, about 30%, 25%, and 20% of HeLa, 4T1, and B16F10 cells underwent pyroptosis after GSDMB^{NT} mRNA@LNPs treatment, as shown in FIG. 2F. The calcein release assays also demonstrate that GSDMB^{NT} mRNA@LNPs treatment caused more cell death compared with either LNPs or GSDMB^{NT} mRNA alone, as shown in FIG. 2E.

GSDMB^{NT} mRNA@LNPs Promote Immunogenic Cell Death

As pyroptosis promotes the release of DAMPs to stimulate immune responses 27, GSDMB^{NT} mRNA@LNPs can induce the release of DAMPs to promote immunogenic cell death (ICD). To confirm this, the expression of ICD biomarkers were measured in cells, including surface-exposed calreticulin (CRT) (“eat me” signal), extracellularly released high mobility group box 1 (HMGB1) (“danger” signal), and adenosine triphosphate (ATP) (“find me” signal) 28.

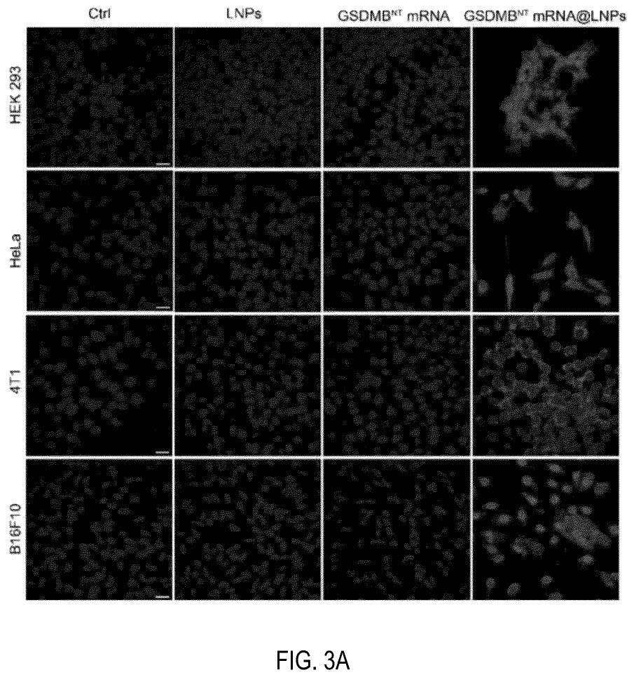

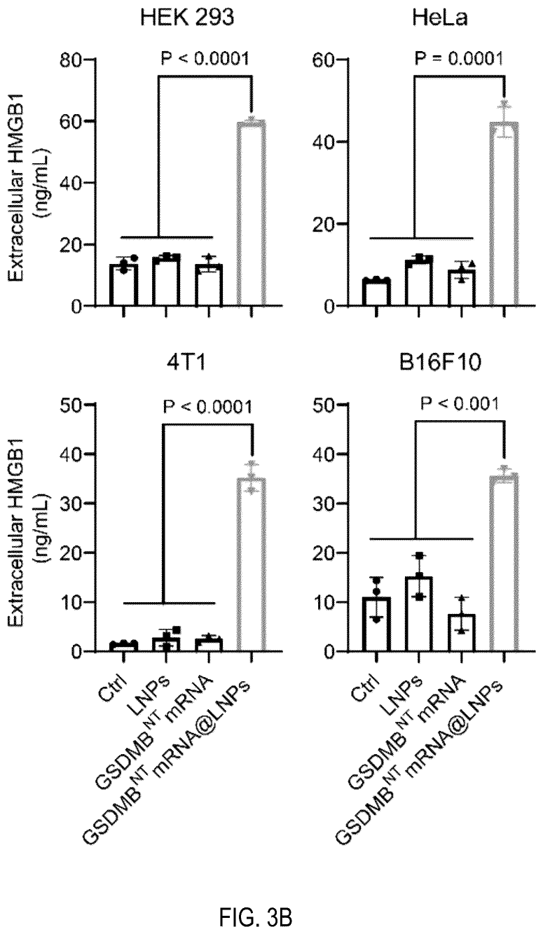

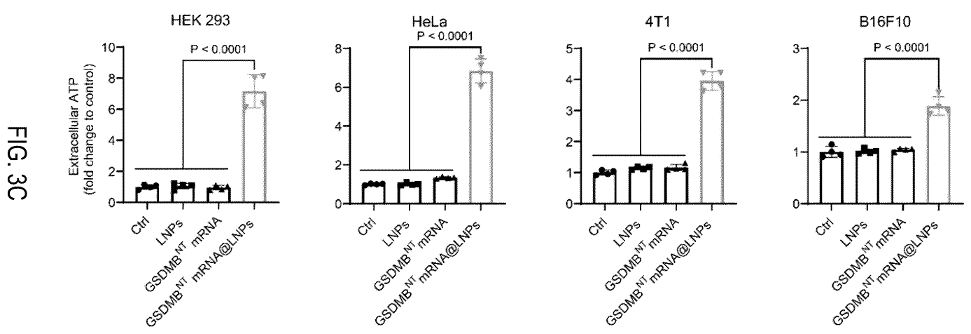

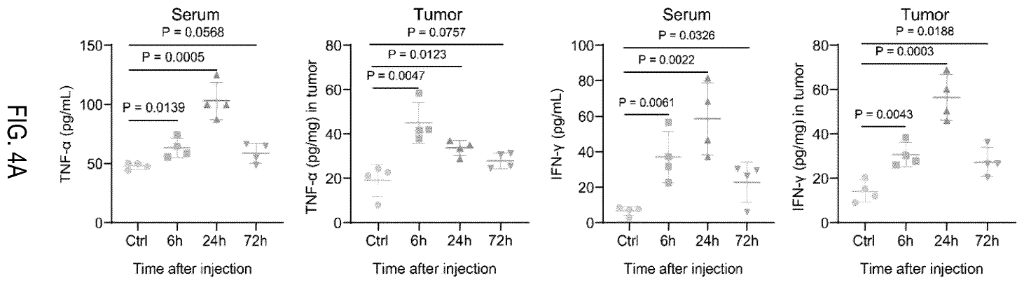

FIG. 3 shows that GSDMB^{NT} mRNA@LNPs promote immunogenic cell death (ICD) in vitro. FIG. 3A shows detection using confocal microscopy of CRT exposure in treated HEK 293, HeLa, 4T1, and B16F10 cells, with a scale bar of 20 μm. FIGS. 3B-3C show extracellular HMGB1 and ATP expression as analyzed by ELISA in HEK 293, HeLa, 4T1, and B16F10 cells after different treatments.

As shown in FIG. 3A, cells incubated with LNPs alone or GSDMB^{NT} mRNA alone had almost no CRT expression, while significant CRT signals were observed in GSDMB^{NT} mRNA@LNPs transfected cells. Compared to controls, HMGB1 and ATP levels were significantly increased in all four cell lines after treatment with GSDMB^{NT} mRNA@LNPs for 24 hours (FIGS. 3B-C). Altogether, these data suggest that less than 40% of cell pyroptosis induced by GSDMB^{NT} mRNA@LNPs was adequate to trigger robust ICD in cancer cells.

### Example 3: Treatment with GSDMB^{NT} mRNA@LNPs Improves Tumor Control in an αPD-1-Resistant 4T1 Breast Cancer Mouse Model

As reported previously, mice bearing 4T1 breast carcinoma are resistant to immune checkpoint inhibitors, such as anti-PD-1 (aPD-1) therapy 29. To investigate the translation efficacy of synthesized mRNA after intratumoral administration, the present inventors transcribed two kinds of firefly luciferase-encoding mRNAs with different capping methods, Anti-Reverse Cap Analog (ARCA)-capped luciferase mRNA and CleanCap-capped luciferase mRNA. Subsequently, to evaluate the in vivo transfection effect of LNPs, these two engineered mRNAs were encapsulated into LNPs to form ARCA-capped Luc mRNA@LNPs or CleanCap-capped Luc mRNA@LNPs. The fabricated Luc mRNA@LNPs were intratumorally injected into an orthotopic 4T1 breast tumor model and luciferase activity was analyzed by in vivo bioluminescent imaging after 6 hours.

### Animals and Mouse Tumor Models

Balb/c female mice and C57BL/6 female mice aged 6-8 weeks were purchased from the Jackson Laboratory and housed in a temperature-controlled environment on a 12-hour light cycle with free access to food and sterile water. All mice were allowed to acclimate for at least 3 days before tumor cell implantation. For an orthotopic breast tumor model, 5×10⁵ 4T1 cells in 50 μL of sterile PBS were injected into the fat pad of the fourth pair of the left breast of Balb/c female mice. B16F10-Luc cells (5× 10⁵) in 100 μL of sterile PBS were implanted subcutaneously into the right flank of C57BL/6 female mice. For the B16F10 dual-flank tumor model, 5×10⁵ cells were implanted subcutaneously on the left side, and 2.5×10⁵ cells were implanted subcutaneously on the right side on the same day. Tumor volume was measured every two days using a Vernier caliper and calculated as V=(a×b²)/2, where a is the long axis and b is the short axis.

### In Vivo Imaging and Drug Administration

Luciferase signals were analyzed by an in vivo imaging system (IVIS, PerkinElmer) to examine the in vivo translation efficiency of mRNA encoding firefly luciferase and monitor the growth of B16F10-Luc-bearing tumor models. Briefly, 100 μL IVISbrite d-Luciferin potassium salt bioluminescent substrate (15 mg/mL in PBS) was injected intraperitoneally. After 10 minutes, mice were imaged in the imaging system. Luminescence intensity was quantified using the living image software (PerkinElmer).

For tumor therapy, one week after tumor inoculation, mice were received with PBS, LNPs, GSDMB^{NT} mRNA@LNPs, or aPD-1 as indicated. Briefly, 50 μL of GSDMB^{NT} mRNA@LNPs was administered intratumorally into the tumors on days 7, 10, 13, and 16 for a total of four doses. Anti-mouse PD-1 antibodies (Bio X Cell, clone RMP1-14) were injected intraperitoneally at a dose of 100 μg on days 8, 11, 14, and 17 for a total of four doses. During the study, mice were checked daily for adverse clinical reactions. The body weight of mice was monitored every two days until the end of the experiments.

### ATP, HMGB1, and Cytokines Detection

HEK 293, Hela, 4T1, and B16F10-Luc cells were seeded in 12-well plates and were incubated with GSDMB^{NT} mRNA, LNPs, or GSDMB^{NT} mRNA@LNPs. After a treatment within 48 hours, the cell culture medium was collected for extracellular ATP assay (Promega) and extracellular HMGB1 analysis (Chondrex). To evaluate intratumoral ATP, HMGB1, and cytokines including TNF-α and IFN-γ, tumor tissues were excised and homogenized in tissue extraction reagents including 1% proteinase and phosphatase inhibitors (Thermo Fisher Scientific). The supernatant from tumor homogenates was then measured with ELISA kits (cytokine kits purchased from Thermo Fisher Scientific).

### Western Blot Analysis

Protein expressions were evaluated using Western blot methods. The Western blot methods have been previously reported⁴⁴. Briefly, the cell samples were treated with Radioimmunoprecipitation (RIPA) lysis buffer containing protease inhibitors to extract the proteins in an ice-cold bath. The supernatant was collected and measured by bicinchoninic acid (BCA) protein assay kit (Thermo Fisher Scientific) to evaluate the protein concentration. Individual samples with equivalent amounts of protein were resolved by sodium dodecyl-sulfate polyacrylamide gel electrophoresis (SDS-PAGE), and the bands were transferred to polyvinylidene fluoride (PVDF) membrane and then blocked with 5% milk. Membranes were incubated with primary antibodies overnight. After washing three times with TBST (20 mM Tris, 160 mM NaCl, 0.1% Tween 20), the membranes were incubated with a secondary antibody for 2 hours at 37° C. After washing three times with TBST, the ChemiDoc XRS system (Bio-Rad) was used to detect the chemiluminescent signals.

### Flow Cytometry for Immune Cells and Antibodies

Purified anti-mouse CD16/32 antibody (clone 93), PerCP/Cyanine5.5 anti-mouse CD45.2 Antibody (clone 104), APC anti-mouse CD11c Antibody (clone N418), FITC anti-mouse I-A/I-E Antibody (clone M5/114.15.2), APC anti-mouse CD3 Antibody (clone 17A2), FITC anti-mouse CD4 Antibody (clone GK1.5), PE anti-mouse CD8a Antibody (clone 53-6.7), Alexa Fluor® 647 anti-mouse FOXP3 Antibody (clone MF-14), PE anti-mouse CD25 Antibody (clone 3C7), FITC anti-mouse NK-1.1 Antibody (clone PK136), FITC anti-mouse/human CD11b Antibody (clone M1/70), APC anti-mouse Ly-6C Antibody (clone HK1.4) and PE/Cyanine7 anti-mouse Ly-6G Antibody (clone 1A8) were purchased from BioLegend.

The expression of stimulatory markers of dendritic cells (CD11c⁺ and major histocompatibility complex II⁺ (MHCII⁺), natural killer (NK) cells (CD3-NK1.1+), NK T cells (CD3+NK1.1+), and monocytes (CD11b⁺Ly6g⁻Ly6c⁺) were analyzed by fluorescence-activated single cell sorting (FACS). Briefly, tumors and lymph nodes were harvested and digested by 1 mg/mL collagenase IV (Thermo Fisher Scientific) for 30 minutes at 37° C. to make single-cell suspensions. The single-cell suspensions were then passed through 70 μm nylon cell strainers. The suspensions were centrifuged, and the cell pellets were washed and resuspended in the PBS containing 1% BSA (FACS buffer), blocked with anti-mouse CD16/CD32 for 30 minutes, and finally stained with the indicated antibodies for another 1 hour. Stained samples were analyzed using a FACS analyzer (BD Biosciences, San Jose, CA). All flow cytometry data were analyzed using FlowJo software.

### Immunofluorescence, Histopathology, and In Vivo Pyroptosis Assay for Tissues

At the end point of treatment, the tumors and organs were harvested and embedded in OCT tissue cassettes and frozen on dry ice for sectioned into slices at a thickness of 10 μm. For tissue immunofluorescence, sample sections were fixed with 4% paraformaldehyde for 30 minutes and then permeabilized with 1% Triton X-100 PBS (PBST) and blocked with a PBST blocking solution containing 5% goat serum for 1 hour at room temperature. Next, the sample slides were incubated with diluted antibodies and imaged as described in the “cell immunofluorescence” section. For histopathology analysis, tissue sections with 10 μm thickness were stained with hematoxylin and eosin (H&E) for pathology following the manufacturer's instructions. To evaluate the tumor cell pyroptosis in vivo, mice were intravenously treated with 2.5 mg/kg propidium iodide. After 20 minutes, tumors and major organs were harvested and then embedded into OCT-containing Cryomold molds and frozen for sectioned into slices at a thickness of 10 μm. After slicing and mounting, the tissue sections were imaged directly and immediately on a fluorescence microscope (BZ-X710; Keyence, Kyoto, Japan).

### Statistical Analysis

All results are analyzed using GraphPad Prism software and presented as the means±SD. Unpaired t-test and one-way ANOVA were used for two-group or multiple-group comparisons. The details of statistical analysis for figures and extended data figures are performed as indicated in the figure legends, and survival analysis was analyzed using the log-rank test.

### Results

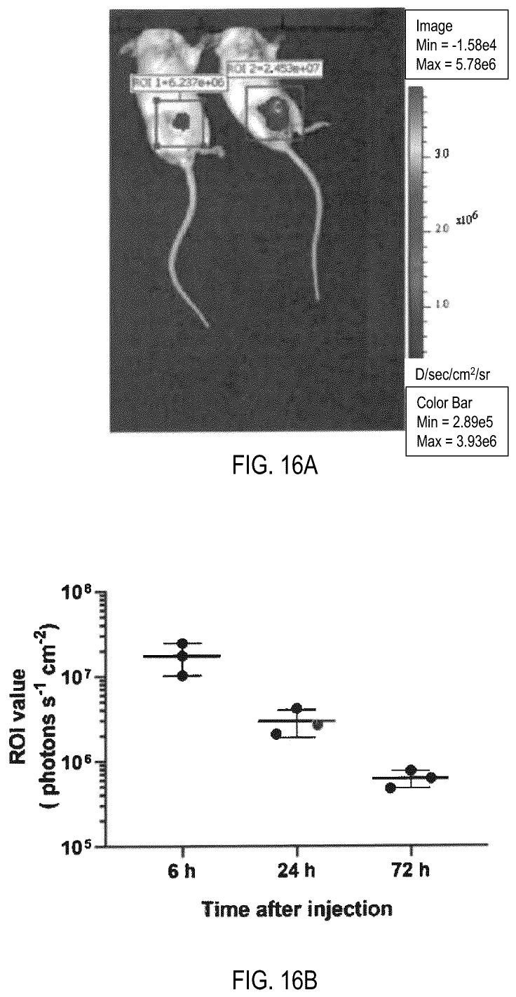

FIG. 16 shows in vivo bioluminescence of luciferase mRNA-encapsulating LNPs of intratumoral injection into orthotopic 4T1 tumors. FIG. 16A shows representative images demonstrating luciferase activity after 6 hours. Anti-reverse cap analog (ARCA)-capped luciferase mRNA (left) and CleanCap capped luciferase mRNA (right) are shown. In FIG. 16B, a time course of CleanCap capped luciferase mRNA bioluminescence activity is shown as photons s⁻¹ cm⁻² as values within regions of interest (ROIs).

As shown in FIG. 16A, a four-time higher bioluminescence intensity was observed in the tumors treated with CleanCap-capped Luc mRNA@LNPs. Almost no luciferase signal was detected in major organs, which indicated that the majority of the Luci mRNA@LNP had accumulated in the tumors. Thus, GSDMB^{NT} mRNA@LNPs with CleanCap-capping were used for further animal experiments. Moreover, a single injection of CleanCap-capped Luc mRNA@LNPs maintained elevated levels of the bioluminescence signal for 3 days in 4T1 tumor sites, as shown in FIG. 16B. These findings suggest that the mRNA/LNPs delivery system of the present invention can transfect cells for a fast, robust, and durable gene expression in vivo.

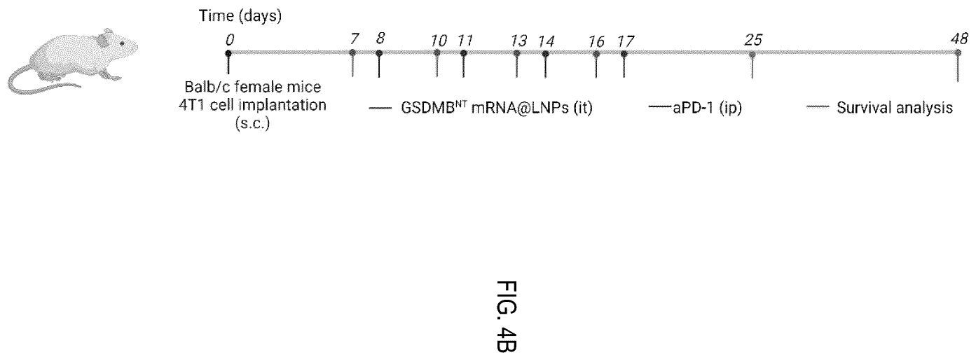

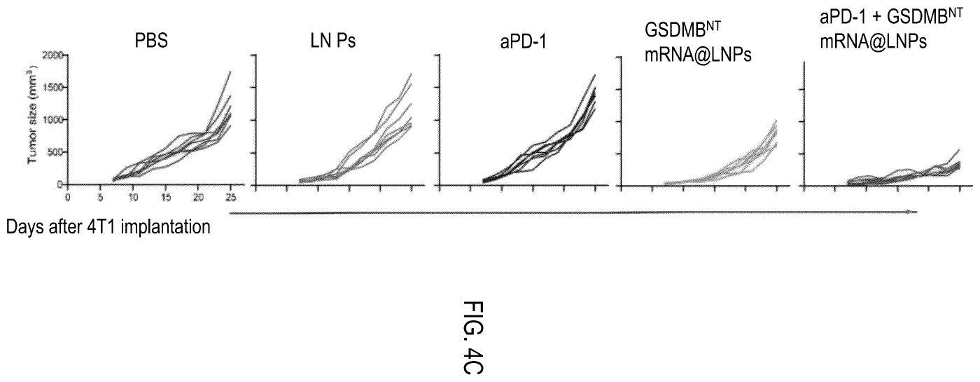

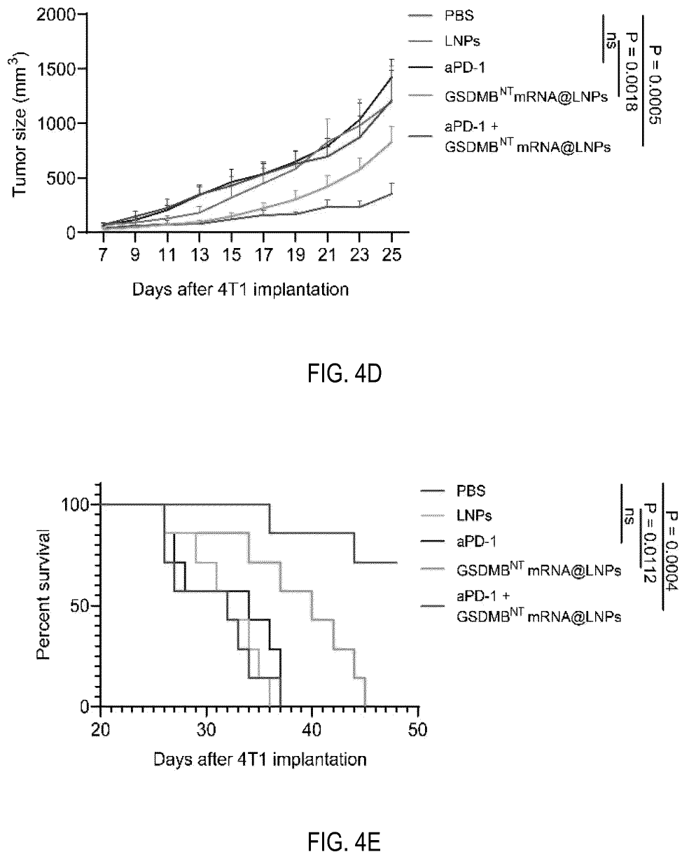

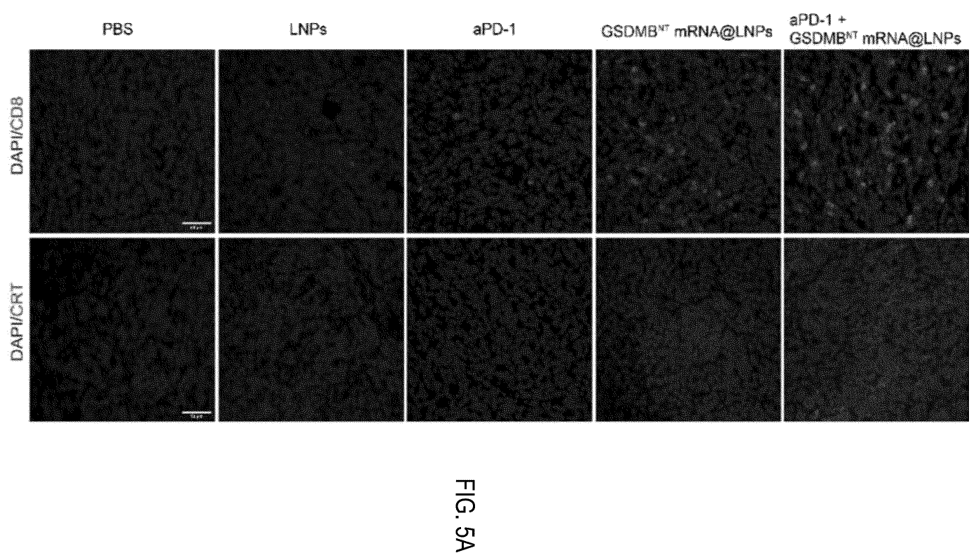

To explore the therapeutic efficacy of intratumorally administered GSDMB^{NT} mRNA@LNPs, antitumor immune responses were first evaluated in the orthotopic 4T1 breast tumor model. FIG. 4 shows that treatment with GSDMB^{NT} mRNA@LNPs promoted tumor control in an anti-PD-1 resistant 4T1 breast cancer mouse model. FIG. 4A shows cytokine concentrations measured in tumor tissue or serum by ELISA, at 6, 24, and 72 hours after a single dose of GSDMB^{NT} mRNA@LNPs (40 μg mRNA). FIG. 4B shows the experimental timelines for treatment of 4T1 orthotopic tumor-bearing mice. s.c.=subcutaneous; it=intratumoral; ip=intraperitoneal. FIG. 4C shows individual growth curves of tumor sizes for mice treated as indicated. FIGS. 4D-E show the average tumor growth curves and survival percentages for mice treated as indicated. Data are represented as means+/standard deviation (SD), with n=7 mice for each group. n.s. indicates no significant difference.

40 μg of GSDMB^{NT} mRNA@LNPs was intratumorally injected into 4T1 tumors and then the concentrations of inflammatory cytokines including tumor necrosis factor-alpha (TNF-α) and interferon-gamma (IFN-γ) in tumor tissue or serum were assayed by ELISA after 6, 24, or 72 hours, separately, as shown in FIG. 4A. Both TNF-α and IFN-γ in serum and tumor increased after 6 hours of GSDMB^{NT} mRNA@LNPs treatment and maintained elevated levels for 3 days. The antitumor efficacy of GSDMB^{NT} mRNA@LNPs was chosen for further exploration by in vivo studies.

To further investigate whether GSDMB^{NT} mRNA@LNPs could sensitize immunological “cold” tumors to ICB-mediated therapy, the present inventors evaluated the antitumor activity of combinatorial treatments of GSDMB^{NT} mRNA@LNP and aPD-1 in orthotopic 4T1 breast cancer models following the treatment timeline shown in FIG. 4B. Briefly, wild-type female Balb/c mice were inoculated subcutaneously with ˜5×10⁵ 4T1 tumor cells into the fourth mammary fat pad and then intratumorally administered on days 7, 10, 13, and 16 with PBS, LNPs, or GSDMB^{NT} mRNA@LNPs (with 10 μg GSDMB^{NT} mRNA). aPD-1-treated mice received intraperitoneal administration of anti-PD-1 antibodies (100 μg per mouse) on days 8, 11, 14, and 17. Tumor growth was recorded by tumor volume measurements taken every two days and the survival monitoring ended at day 48 (30 days after the final treatment). After four rounds of treatments, the results showed that tumors grew rapidly in PBS, LNPs, and aPD-1-treated groups, and all mice died within 37 days, indicating that aPD-1 did not inhibit tumor growth in 4T1 tumors.

In contrast, GSDMB′T mRNA@LNPs effectively inhibited tumor growth (P=0.0018) and prolonged animal survival to 45 days. Massive tumor shrinkage (P=0.0005) and a more than 70% survival rate (P=0.0004) occurred in mice treated with the GSDMB^{NT} mRNA@LNPs+aPD-1 combination therapy at day 48, as shown in FIGS. 4C-E. These results indicate that GSDMB^{NT} mRNA@LNPs improved tumor suppression and strengthened the sensitivity of aPD-1 antibody therapy in an aPD-1-resistant 4T1 tumor model.

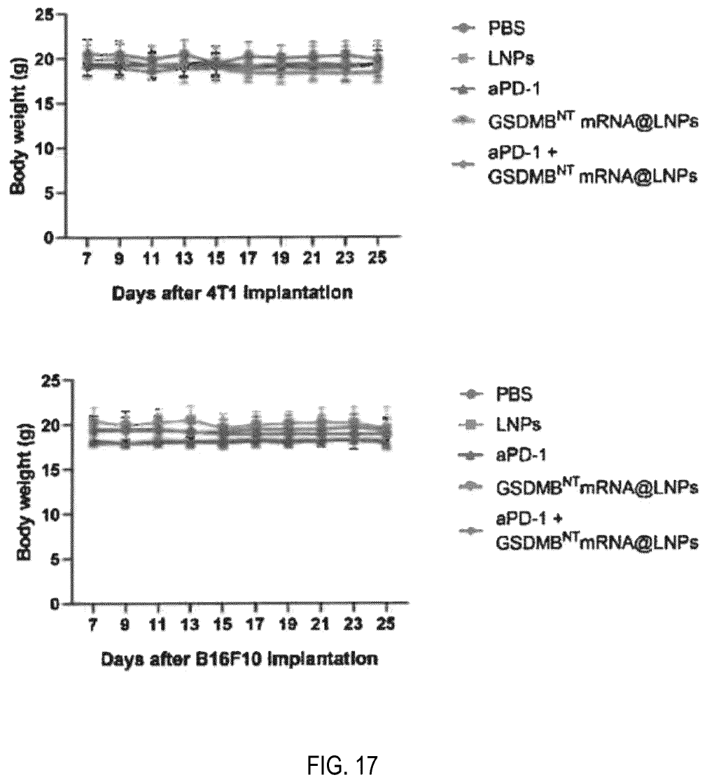

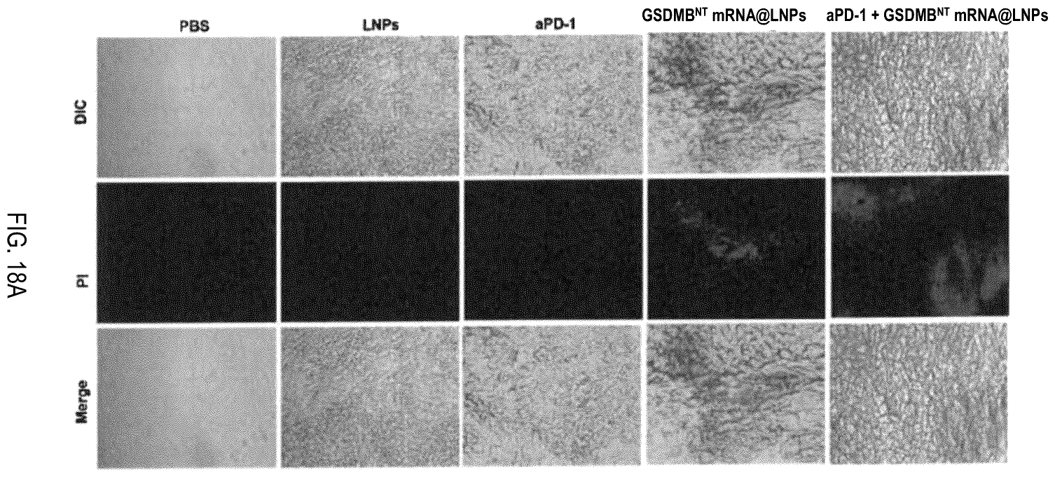

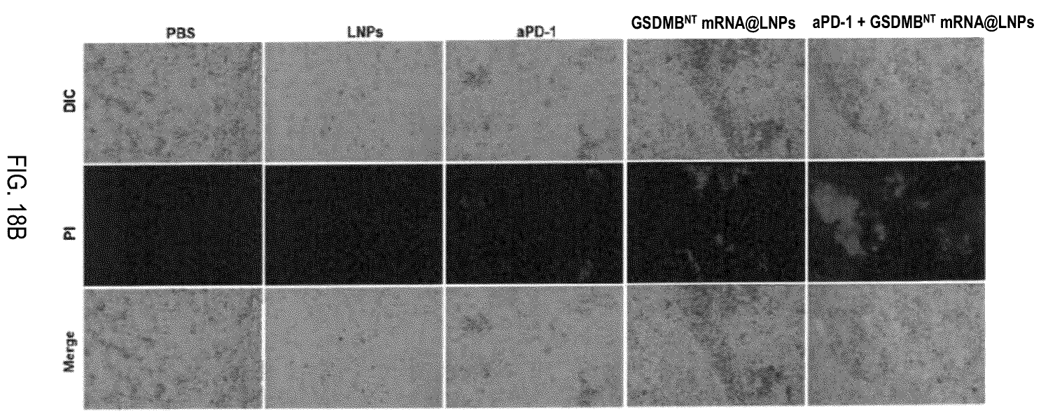

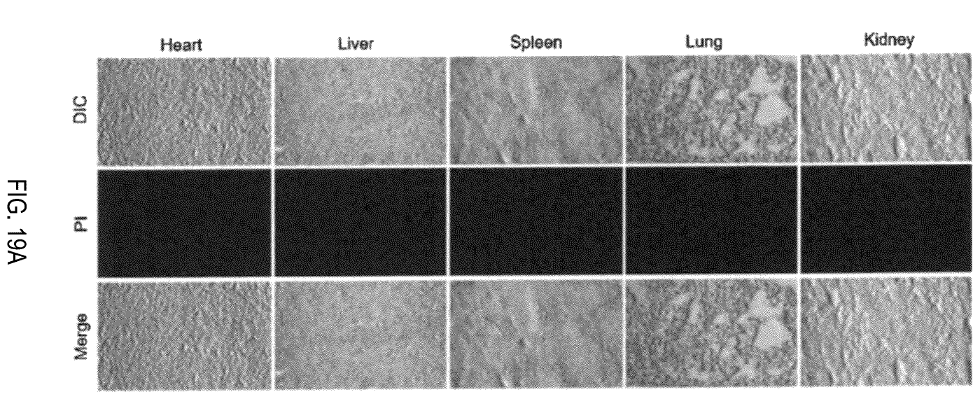

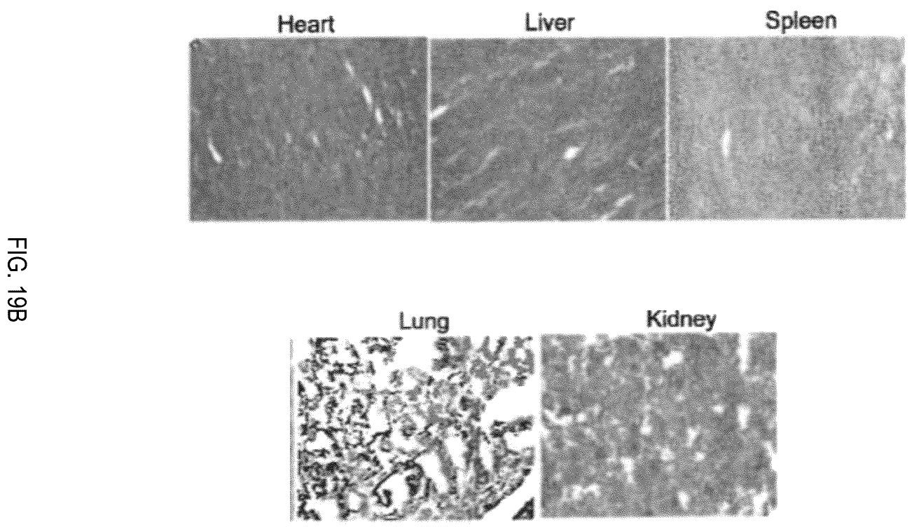

FIG. 17 shows changes in body weight in 4T1-bearing mice (top) and B16F10-bearing mice (bottom) with various treatments. FIG. 18 shows representative tumor-section images of in vivo pyroptosis cells in the orthotopic 4T1-bearing tumor model (FIG. 4A) and B16F10-bearing tumor model (FIG. 4B). Propidium iodide was intravenously injected into the mice before the assay.

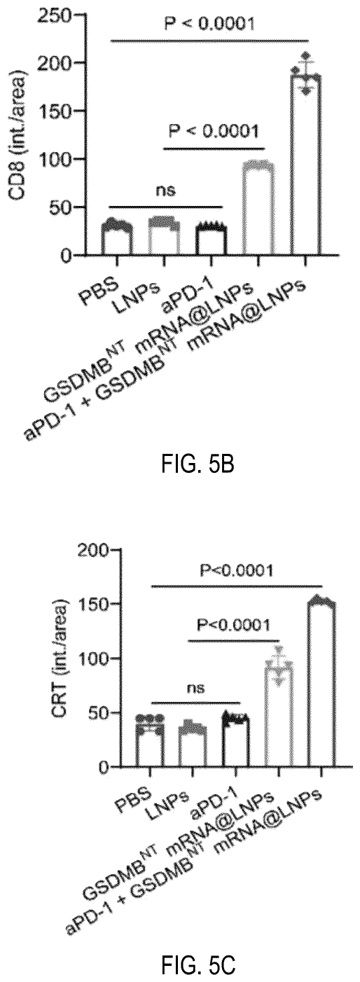

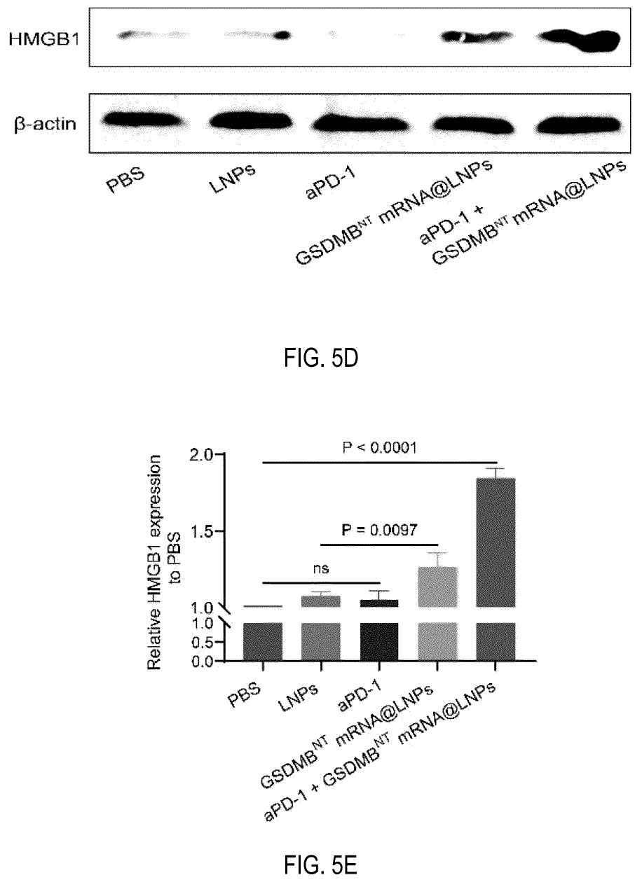

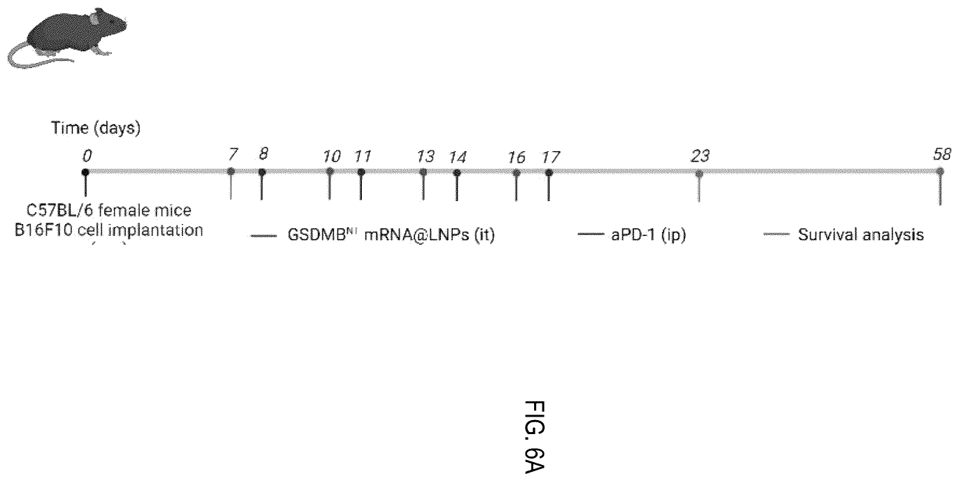

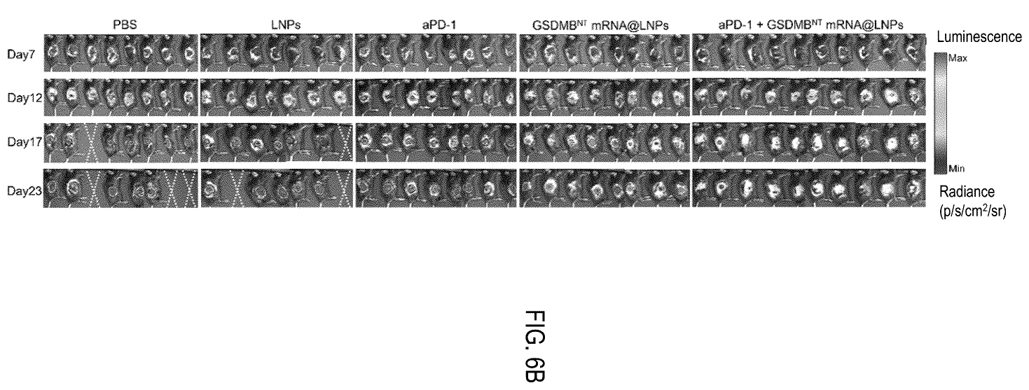

FIG. 5 shows treatment with GSDMB^{NT} mRNA@LNPs caused inducement of ICD in an anti-PD-1-resistant 4T1 breast cancer mouse model. FIG. 5A shows immunofluorescence staining of tumors for CD8⁺ T cell infiltration and CRT expression after the indicated treatments. FIGS. 5B-C show quantitative analysis of immunofluorescence staining in terms of CD8⁺ and CRT intensities (n=5). FIGS. 5D-E show imaging and quantification of western blotting of HMGB1 in the supernatant of tumors excised from mice treated as indicated. β-actin was used as a housekeeping standard. n.s. indicates no significant difference.

In addition, immunofluorescence and western blot analysis of tumor tissues showed increased CD8, CRT, and HMGB1 expression in the GSDMB^{NT} mRNA@LNP group compared with the control groups. After the GSDMB^{NT} mRNA@LNPs+aPD-1 combination treatment, CD8, CRT, and HMGB1 expression increased by 6.7-, 4.3-, and 1.9-fold compared with the control groups. However, almost no changes in these DAMPs were detected in the aPD-1 group, as shown in FIGS. 5A-E. Similar results were observed in the evaluation of PI-positive cells in tumor tissues, as shown in FIG. 18A. Furthermore, mice treated with GSDMB^{NT} mRNA@LNPs did not show weight loss or abnormalities in major organs, as shown in FIG. 17. These results demonstrate that GSDMB^{NT} mRNA@LNPs exert antitumor immunity accompanied by the release of DAMPs in vivo.

Treatment with GSDMB^{NT} mRNA@LNPs Remodels the Tumor Microenvironment and Enhances Potent Antitumor Activity in an Aggressive B16F10 Melanoma Mouse Model

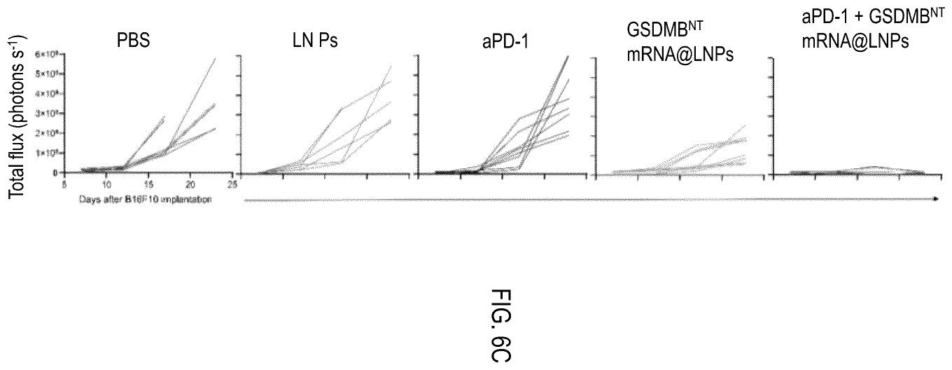

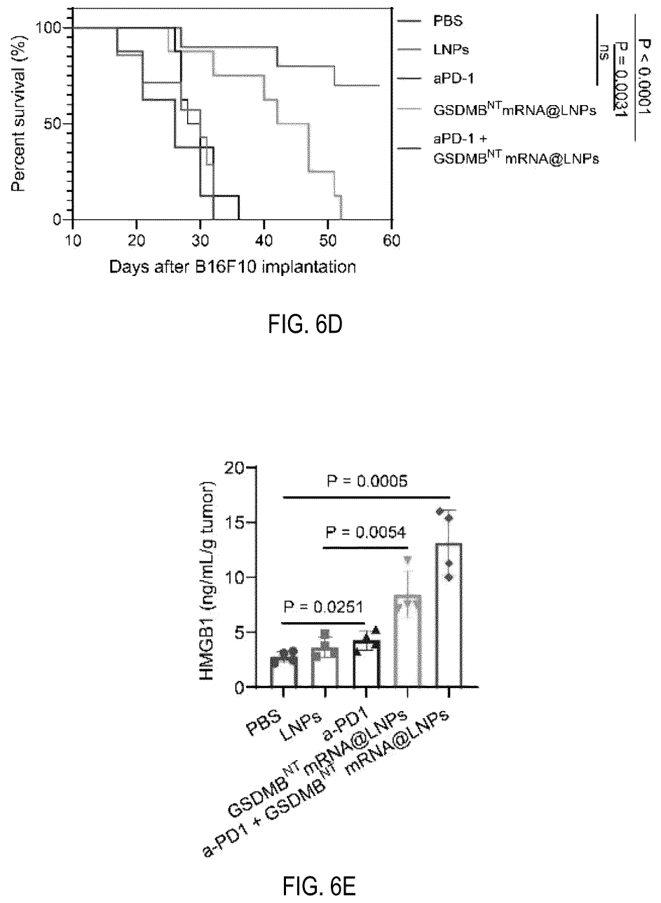

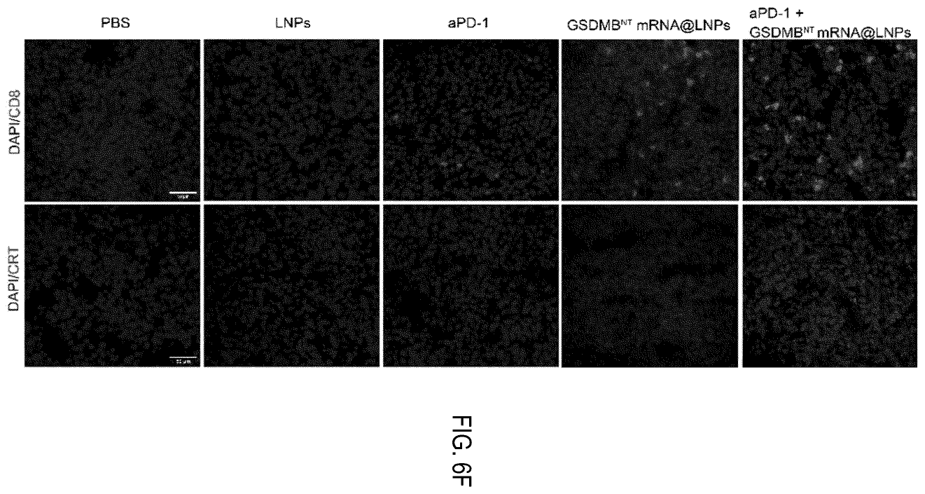

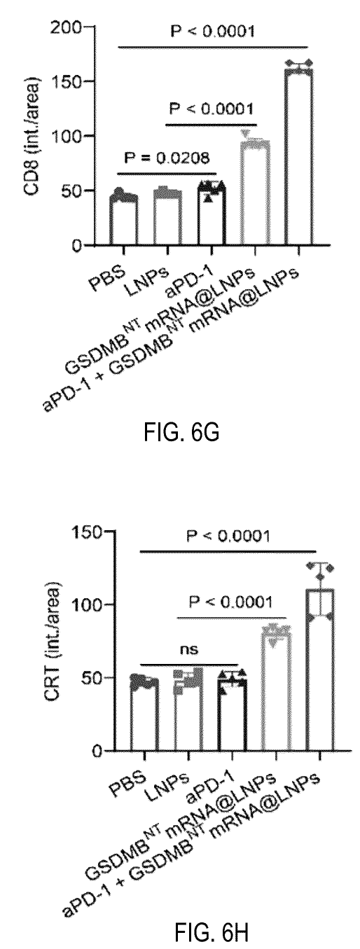

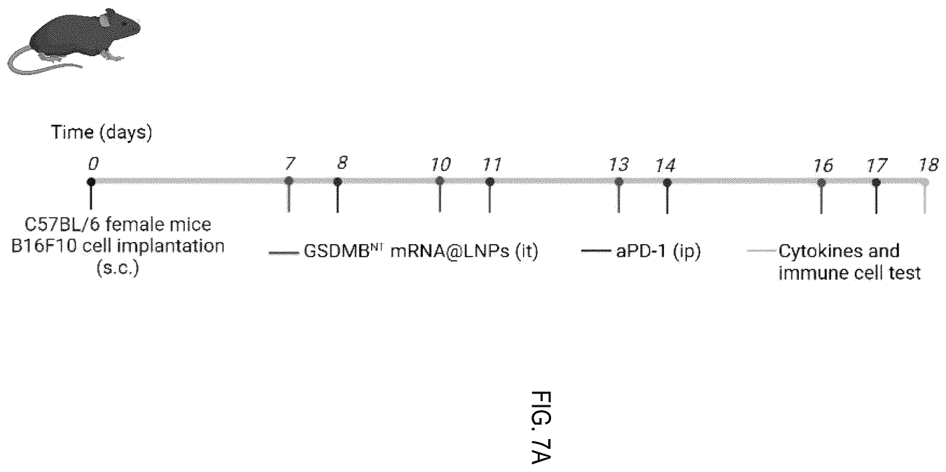

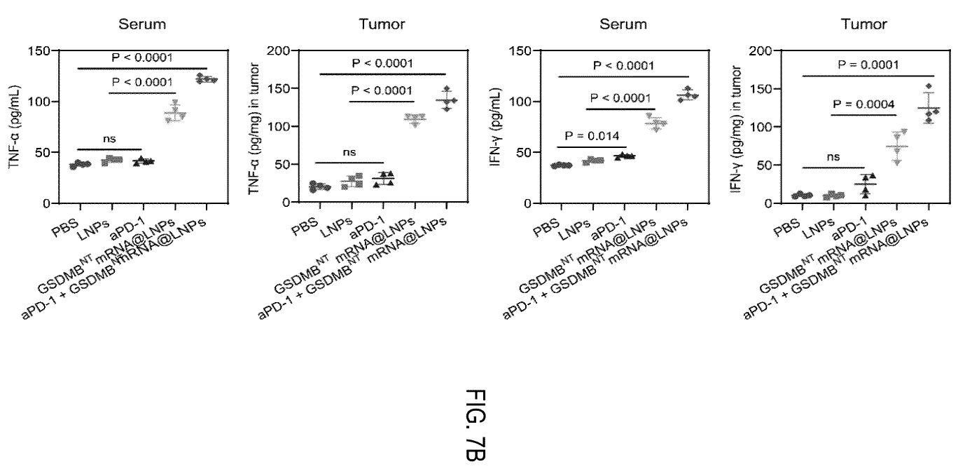

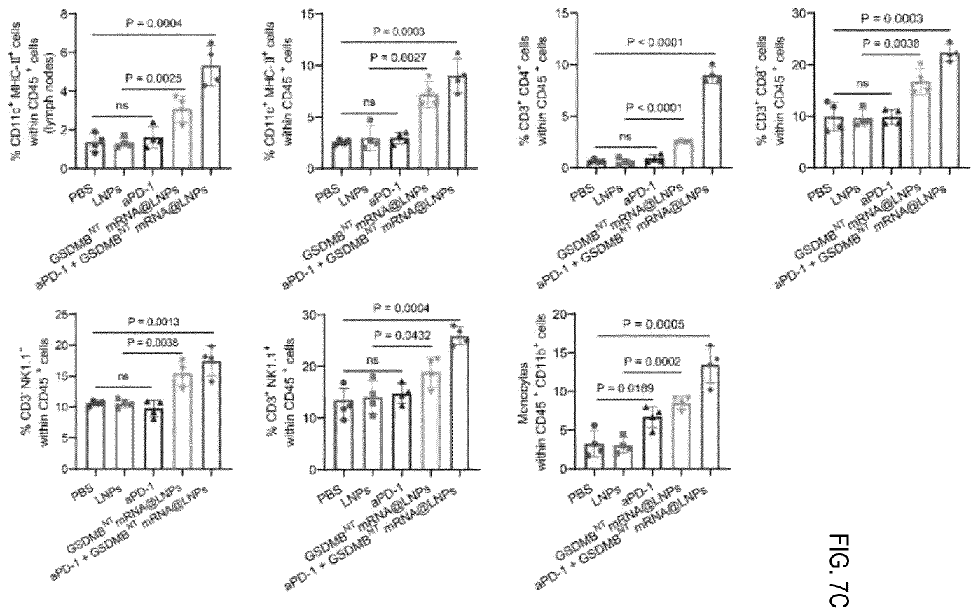

FIG. 6 demonstrates that treatment with GSDMB^{NT} mRNA@LNPs enhanced potent antitumor activity in an aggressive melanoma mouse model. FIG. 6A shows the experimental timeline for treatment of B16F10 tumor-bearing mice. FIGS. 6B-C show in vivo bioluminescence images and quantification of luciferase signals in mice treated as indicated for monitoring tumor growth. The imaging was performed every 5 days from the initial treatment (day 7 after tumor inoculation) until day 23. FIG. 6D shows the survival percentages of the mice treated as indicated. n=8 mice for PBS, aPD-1, or GSDMB^{NT} mRNA@LNPs treatment groups; n=7 mice for LNPs treatment group; and n=10 for aPD-1 +GSDMB^{NT} mRNA@LNPs treatment group. FIG. 6E shows ELISA analysis of HMGB1 in the supernatant of B16F10 tumors excised from mice treated as indicated. The data are presented as means+/−standard deviation (SD), with n=4 mice per group. FIGS. 6F-H show representative images and quantitative analysis of immunofluorescence staining for CD8⁺ T cell infiltration and CRT expression in tumors after the indicated treatments (n=5). The results are presented as means+/−standard deviation (SD). n.s. indicates no significant difference.

To further verify the synergistic effect of GSDMB^{NT} mRNA@LNPs in immunotherapy, the present inventors investigated the antitumor activity in an aggressive melanoma mouse model. GSDMB^{NT} mRNA@LNPs and aPD-1 antibodies were administered in B16F10 tumor models following the timeline as shown in FIG. 6A. Briefly, 5×10⁵ B16F10-Luc cells were implanted subcutaneously on the right flank of the C57BL/6 female mice to establish subcutaneous tumors. 7 days after cell implantation, mice were intratumorally injected with PBS, LNPs, or GSDMB^{NT} mRNA@LNPs (with 10 μg GSDMB^{NT} mRNA) on days 7, 10, 13, and 16. aPD-1-treated mice received intraperitoneal administration of anti-PD-1 antibodies (100 μg per mouse) on days 8, 11, 14, and 17. Tumor imaging was carried out every 5 days for a total of 4 times from initial treatment and survival analysis ended at day 58 (40 days after the final treatment).

In FIGS. 6B-C, the images and quantitative analysis of bioluminescence signals showed that tumors grew rapidly in control groups, and 3/8 (PBS-treated) and 2/7 (LNP-treated) mice died within 23 days. The aPD-1 alone treatment exhibited an antitumor effect at early time points but failed to achieve sustained tumor inhibition. The survival time of aPD-1-treated mice was slightly prolonged from 32 days in PBS and LNP-treated mice to 36 days, indicating that aPD-1 is not effective in suppressing tumors.

In contrast, mice treated with GSDMB^{NT} mRNA@LNPs showed superior tumor inhibition and extended survival time (52 days). Especially, the combinational therapy of GSDMB^{NT} mRNA@LNPs and aPD-1 showed that established tumors were eliminated in 7 of 10 mice and 70% of mice still survived (P<0.0001) at the predetermined endpoint (day 58), as shown in FIG. 6D. Consistently, compared to mice treated with aPD-1 alone, increased CD8, CRT, and HMGB1 expression and PI-positive cells were measured in mice treated with combinational therapy, as shown in FIGS. 6E-H and FIG. 18B. These findings demonstrated that GSDMB^{NT} mRNA@LNPs play an important role in improving the therapeutic efficacy of aPD-1-mediated immunotherapy.

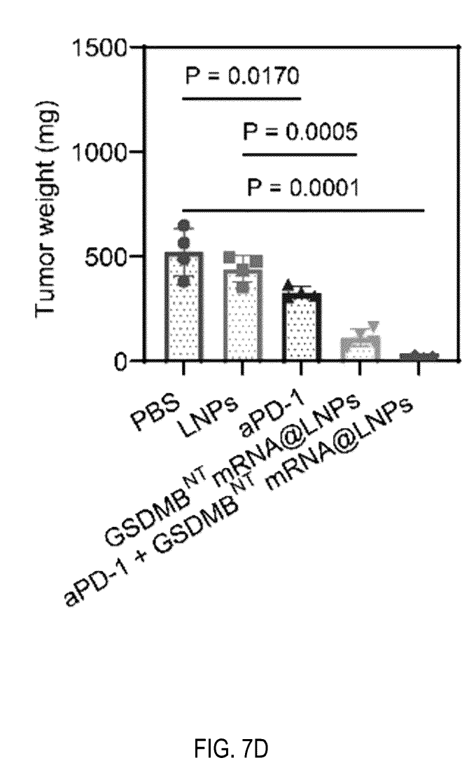

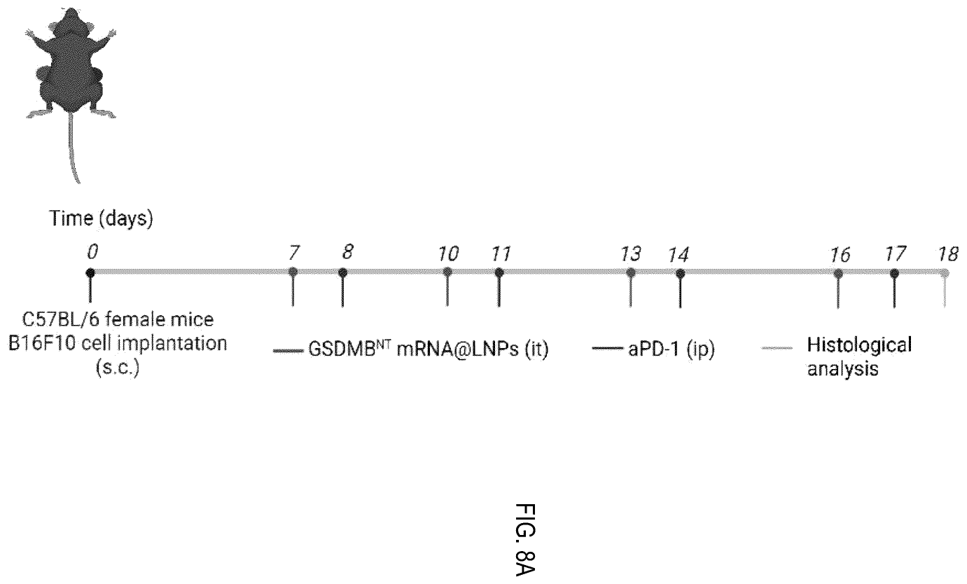

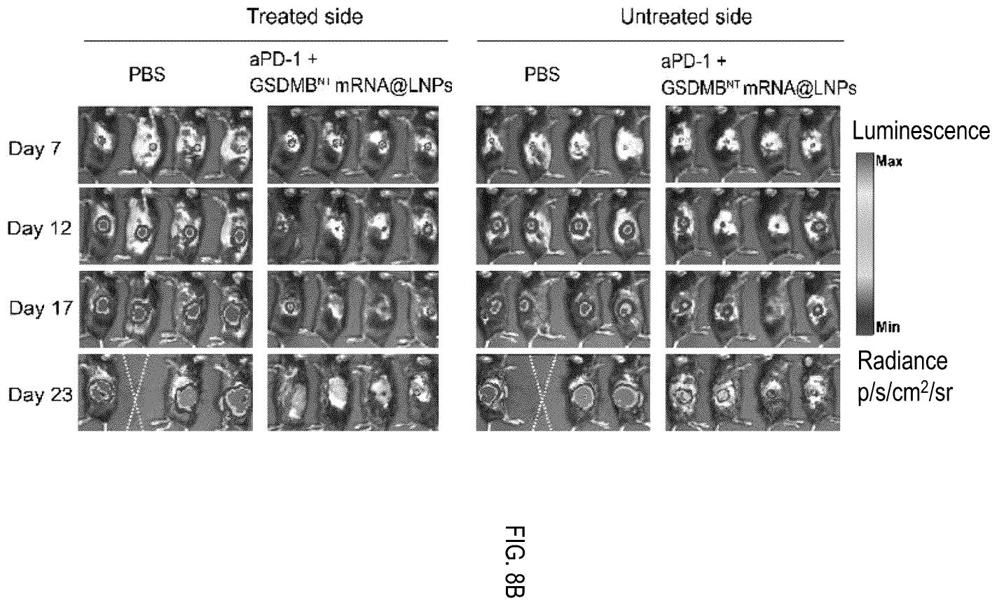

FIG. 7 shows treatment with GSDMB^{NT} mRNA@LNPs can remodel the tumor environment in an aggressive melanoma mouse model. FIG. 7A shows experimental timeline for the treatment of B16F10 tumor-bearing mice, with cytokines and immune cell tests conducted on day 18. FIG. 7B shows cytokine concentrations as measured in tumor tissue or serum by ELISA. FIG. 7C shows flow cytometry analysis results as the percentage of CD11c⁺ MHC-II⁺ dendritic cells, CD3⁺ CD4⁺ T cells, CD3⁺ CD8⁺ T cells, CD3-NK1.1⁺ T cells, CD3⁺ NK1.1⁺ T cells, and monocytes isolated from lymph nodes or tumors. FIG. 7D shows tumor weights of B16F10 tumor-bearing mice with different treatments. Results are presented as means+/−standard deviation (SD) with n=4 mice per group. n.s. indicates no significant difference.

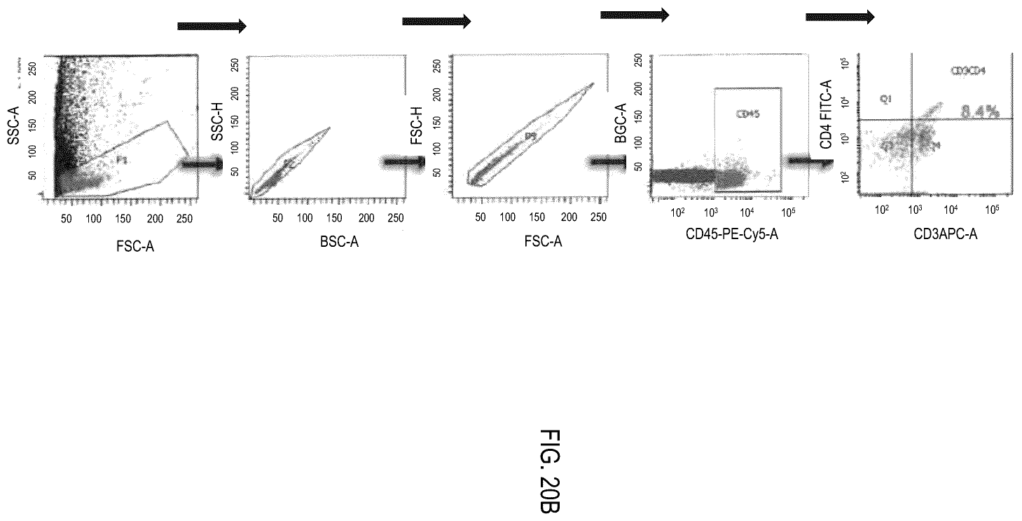

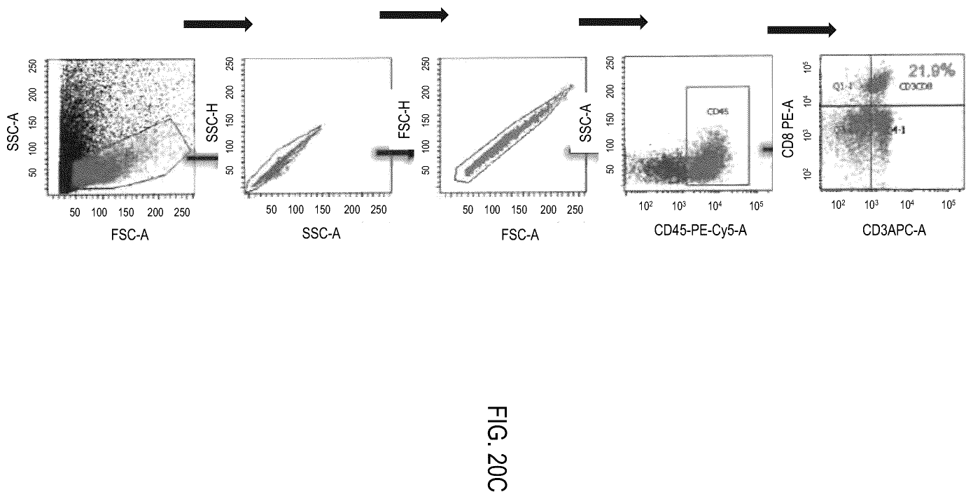

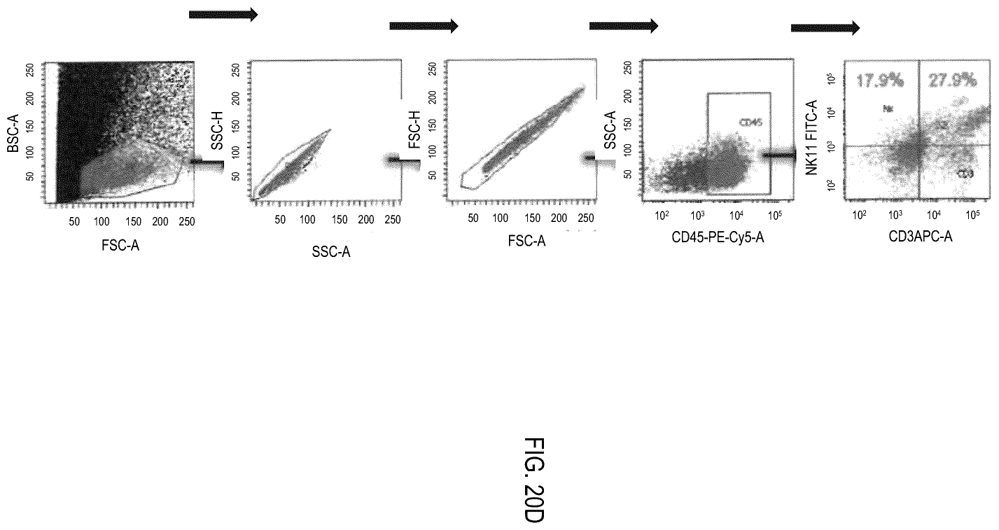

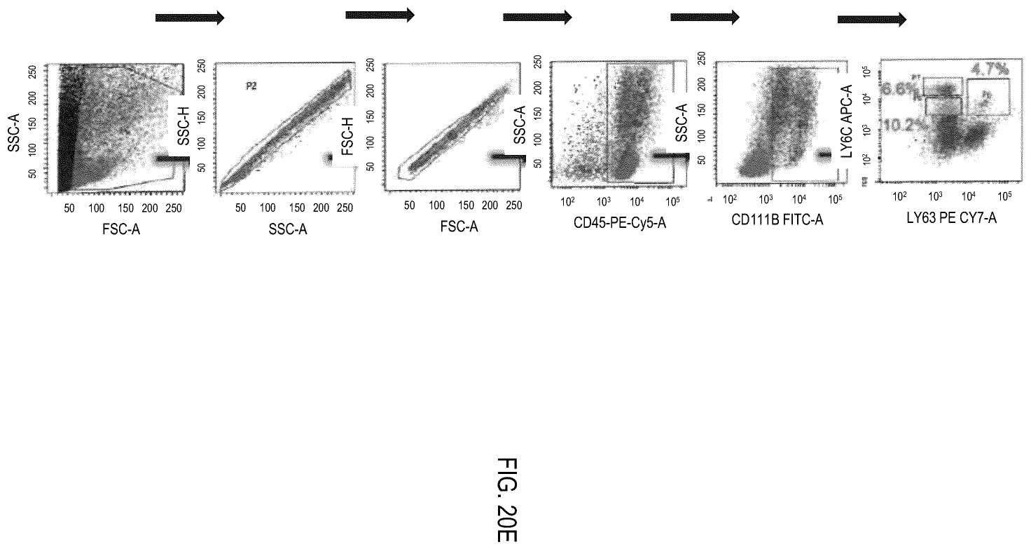

FIG. 20 shows primary gating strategies for flow cytometric analysis of dendritic cells (FIG. 20A), CD4⁺ cells in tumors (FIG. 20B), CD8⁺ cells (FIG. 20C), NK and NK T (Q2) cells (FIG. 20D), granulocytes (P6), M-MDSCs (P7) and monocyte cells (P8) in tumors (FIG. 20E). B16F10 tumor-bearing mice were euthanized, and lymph nodes were isolated on day 18. Singlet cells were selected from the cell population. CD45⁺ cells were selected from the living cell population. MHC=major histocompatibility complex; FSC-A=forward scatter area; SSC-A=side scatter area; FSC-H=forward scatter height; SSC-H=side scatter height.

To better understand how the GSDMB^{NT} mRNA@LNPs remodel the tumor microenvironment, as shown in FIG. 7A, tumors and blood were harvested to assess the concentrations of cytokines on day 18. As shown in FIG. 7B, mice treated with PBS and LNPs alone had similar concentrations of TNF-α and IFN-γ in serum and tumor, while the concentrations of cytokines significantly increased in mice with GSDMB^{NT} mRNA@LNPs treatments. Furthermore, the present inventors identified and characterized different immune cell populations in lymph nodes and tumors using flow cytometry.

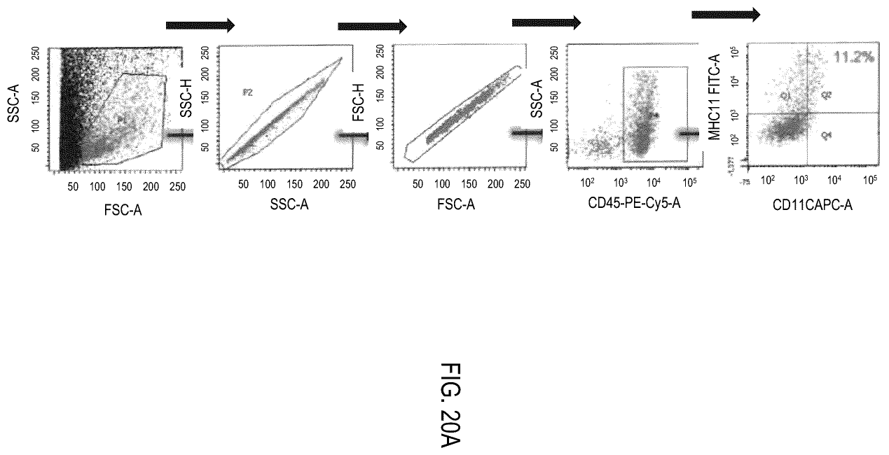

As shown in FIG. 7C and FIG. 20, dendritic cells, CD4⁺ T cells, CD8⁺ T cells, natural killer (NK) cells, and NK T cells were recruited in tumors after the administration of GSDMB^{NT} mRNA@LNPs. Strikingly, compared to the aPD-1 monotherapy group, the population of CD4⁺ T cells showed a 15.6-fold increase in the GSDMB^{NT} mRNA@LNPs+aPD-1 group. Consistently, the tumor weight of mice treated with the combinational therapy was reduced by 17.9-fold compared to that of mice with aPD-1 treatment, as shown in FIG. 7D. Meanwhile, these therapies did not induce body weight loss or cell death and abnormality in major organs, as shown in FIG. 17 and FIG. 19. FIG. 19A shows representative tumor-section images of in vivo pyroptosis cells in major organs in the combinational treatment of aPD-1 and GSDMB^{NT} mRNA@LNPs in B16F10-bearing mice. Propidium iodide was intravenously injected into the mice before the assay. FIG. 19B shows H&E staining of major organs in the combinatorial treatment of aPD-1 and GSDMB^{NT} mRNA@LNPs in B16F10-bearing mice.

These results reveal that GSDMB^{NT} mRNA@LNPs exhibit antitumor immunity by reversing the immunosuppressive TME.

Local Administration of GSDMB^{NT} mRNA@LNPs Enhances Antitumor Immunity in Distant Tumors

To further evaluate whether GSDMB^{NT} mRNA@LNPs can induce systemic immunity against untreated tumors, the present inventors established a B16F10 dual-tumor model that was inoculated with B16F10-Luc cells on the left and right flanks.

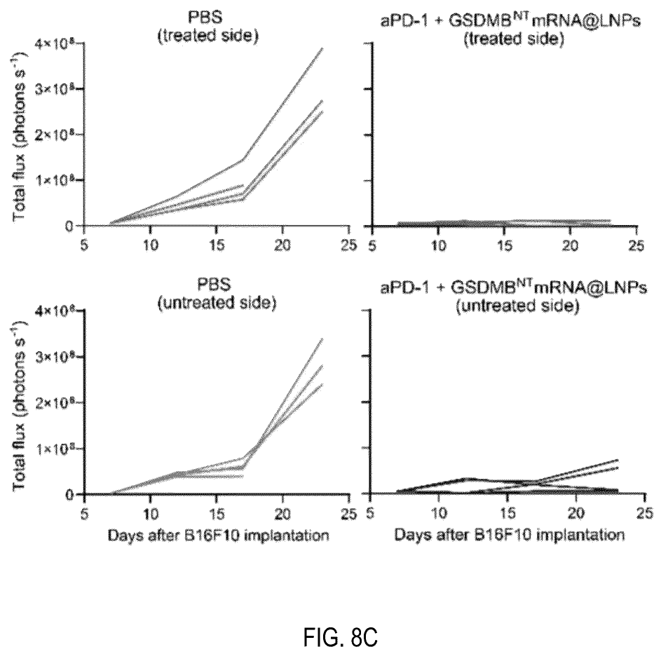

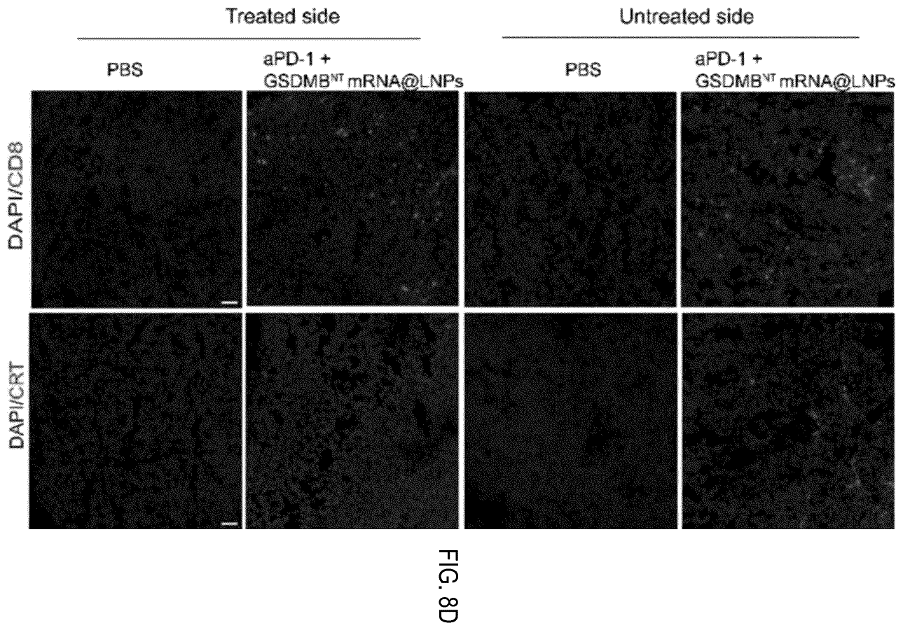

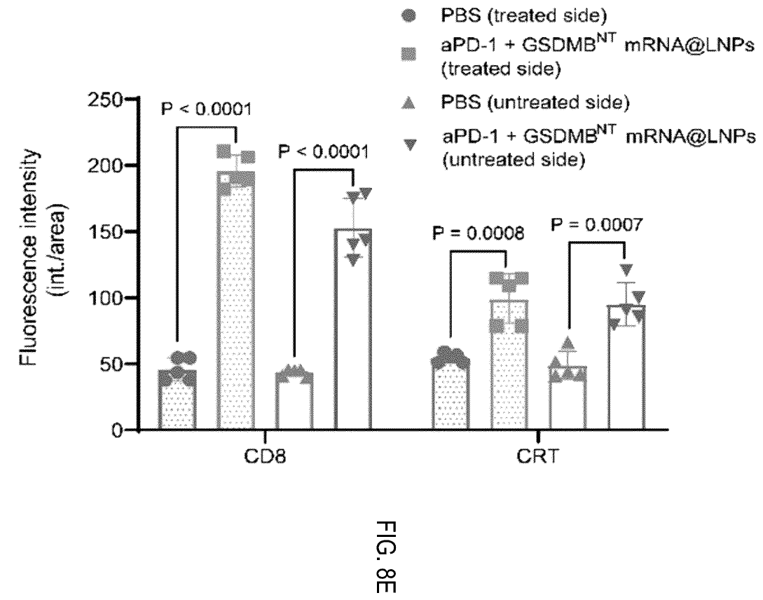

FIG. 8 demonstrates that local combinatorial treatment of GSDMB^{NT} mRNA@LNPs and aPD-1 controlled the tumor burden at distant sites. C57BL/6 mice (n=4 mice per group) were inoculated subcutaneously (s.c.) with 5×10⁵ and 2.5×10⁵ B16F10 cells in the left and right flanks, respectively. FIG. 8A shows the experimental timeline for treatment of B16F10 dual-tumor-bearing mice. FIG. 8B shows in vivo bioluminescence images of luciferase signals in mice treated as indicated for monitoring tumor growth. Imaging was performed every 5 days from the initial treatment day (day 7 after tumor inoculation) until day 23. FIG. 8C shows individual curves of luciferase signals for mice treated as indicated. FIGS. 8D-E show representative images and quantitative analysis of immunofluorescence staining for CD8⁺ T cell infiltration and CRT expression in tumors after indicated treatments (n=5). Results are presented as means+/−standard deviation (SD).

Following the timeline in FIG. 8A, combinational therapy mice were treated with GSDMB^{NT} mRNA@LNPs (with 10 μg GSDMB^{NT} mRNA) on days 7, 10, 13, and 16, and treated with aPD-1 (100 μg per mouse) on day 8, 11, 14, and 17. The in vivo bioluminescence imaging in FIGS. 8B-C revealed that the combinational treatment of aPD-1 antibody and GSDMB^{NT} mRNA@LNPs induced regression of treated tumors and inhibited the growth of untreated tumors. CD8 and CRT expression analysis also confirmed the improvement in the immunosuppressive TME by combinational therapy (FIGS. 8D-E). Together, it was shown that local GSDMB^{NT} mRNA@LNPs treatment not only triggers inflammatory pyroptosis in tumors but also promotes systemic antitumor immunity, which can control tumor growth at remote sites.

While exemplary embodiments have been described herein, it is expressly noted that these embodiments should not be construed as limiting, but rather that additions and modifications to what is expressly described herein also are included within the scope of the invention. Moreover, it is to be understood that the features of the various embodiments described herein are not mutually exclusive and can exist in various combinations and permutations, even if such combinations or permutations are not made express herein, without departing from the spirit and scope of the invention.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

### REFERENCES

1 Riley, R. S., June, C. H., Langer, R. & Mitchell, M. J. Delivery technologies for cancer immunotherapy. Nature reviews Drug discovery 18, 175-196 (2019).
2 Tan, S., Li, D. & Zhu, X. Cancer immunotherapy: Pros, cons and beyond. Biomedicine & Pharmacotherapy 124, 109821 (2020).
3 Fukumura, D., Kloepper, J., Amoozgar, Z., Duda, D. G. & Jain, R. K. Enhancing cancer immunotherapy using antiangiogenics: opportunities and challenges. Nature reviews Clinical oncology 15, 325-340 (2018).
4 Dine, J., Gordon, R., Shames, Y., Kasler, M. K. & Barton-Burke, M. Immune checkpoint inhibitors: an innovation in immunotherapy for the treatment and management of patients with cancer. Asia-Pacific journal of oncology nursing 4, 127-135 (2017).
5 Sharma, P., Hu-Lieskovan, S., Wargo, J. A. & Ribas, A. Primary, adaptive, and acquired resistance to cancer immunotherapy. Cell 168, 707-723 (2017).
6 Topalian, S. L., Drake, C. G. & Pardoll, D. M. Immune checkpoint blockade: a common denominator approach to cancer therapy. Cancer cell 27, 450-461 (2015).
7 Lin, Y.-X. et al. Reactivation of the tumor suppressor PTEN by mRNA nanoparticles enhances antitumor immunity in preclinical models. Science translational medicine 13, eaba9772 (2021).
8 Nam, J. et al. Cancer nanomedicine for combination cancer immunotherapy. Nature Reviews Materials 4, 398-414 (2019).
9 Galon, J. & Bruni, D. Approaches to treat immune hot, altered and cold tumours with combination immunotherapies. Nature reviews Drug discovery 18, 197-218 (2019).
10 Herbst, R. S. et al. Predictive correlates of response to the anti-PD-L1 antibody MPDL3280A in cancer patients. Nature 515, 563-567 (2014).
11 Vanneman, M. & Dranoff, G. Combining immunotherapy and targeted therapies in cancer treatment. Nature reviews cancer 12, 237-251 (2012).
12 Waldman, A. D., Fritz, J. M. & Lenardo, M. J. A guide to cancer immunotherapy: from T cell basic science to clinical practice. Nature Reviews Immunology 20, 651-668 (2020).
13 Agarwal, Y. et al. Intratumourally injected alum-tethered cytokines elicit potent and safer local and systemic anticancer immunity. Nature Biomedical Engineering 6, 129-143 (2022).
14 Chen, D. S. & Mellman, I. Oncology meets immunology: the cancer-immunity cycle. immunity 39, 1-10 (2013).
15 Pio, R., Ajona, D., Ortiz-Espinosa, S., Mantovani, A. & Lambris, J. D. Complementing the cancer-immunity cycle. Frontiers in immunology 10, 774 (2019).
16 Chen, D. S. & Mellman, I. Elements of cancer immunity and the cancer-immune set point. Nature 541, 321-330 (2017).
17 Li, Y. et al. Multifunctional oncolytic nanoparticles deliver self-replicating IL-12 RNA to eliminate established tumors and prime systemic immunity. Nature Cancer 1, 882-893 (2020).
18 Bergsbaken, T., Fink, S. L. & Cookson, B. T. Pyroptosis: host cell death and inflammation. Nature Reviews Microbiology 7, 99-109 (2009).
19 Zhang, Z. et al. Gasdermin E suppresses tumour growth by activating antitumour immunity. Nature 579, 415-420 (2020).
20 Zhou, Z. et al. Granzyme A from cytotoxic lymphocytes cleaves GSDMB to trigger pyroptosis in target cells. Science 368, eaaz7548 (2020).
21 Wang, Q. et al. A bioorthogonal system reveals antitumour immune function of pyroptosis. Nature 579, 421-426 (2020).
22 Lu, Y. et al. Strategies to package recombinant Adeno-Associated Virus expressing the N-terminal gasdermin domain for tumor treatment. Nature Communications 12, 1-14 (2021).
23 Cui, J., Chen, Y., Wang, H. Y. & Wang, R.-F. Mechanisms and pathways of innate immune activation and regulation in health and cancer. Human vaccines & immunotherapeutics 10, 3270-3285 (2014).
24 Lu, L. et al. Emerging mechanisms of pyroptosis and its therapeutic strategy in cancer. Cell Death Discovery 8, 1-13 (2022).
25 Li, Z. et al. Enzyme-Catalyzed One-Step Synthesis of Ionizable Cationic Lipids for Lipid Nanoparticle-Based mRNA COVID-19 Vaccines. ACS nano (2022).
26 Li, Z. et al. Lipid-Polymer Hybrid “Particle-in-Particle” Nanostructure Gene Delivery Platform Explored for Lyophilizable DNA and mRNA COVID-19 Vaccines. Advanced functional materials 32, 2204462 (2022).
27 Walle, L. V. & Lamkanfi, M. Pyroptosis. Current Biology 26, R568-R572 (2016).
28 Yi, Y. et al. Transforming “cold” tumors into “hot” ones via tumor-microenvironment-responsive siRNA micelleplexes for enhanced immunotherapy. Matter (2022).
29 De Henau, O. et al. Overcoming resistance to checkpoint blockade therapy by targeting PI3Ky in myeloid cells. Nature 539, 443-447 (2016).
30 Schumacher, T. N. & Schreiber, R. D. Neoantigens in cancer immunotherapy. Science 348, 69-74 (2015).
31 Xia, X. et al. The role of pyroptosis in cancer: pro-cancer or pro-“host”? Cell death & disease 10, 1-13 (2019).
32 Karki, R. et al. Synergism of TNF-α and IFN-γ triggers inflammatory cell death, tissue damage, and mortality in SARS-COV-2 infection and cytokine shock syndromes. Cell 184, 149-168. e117 (2021).
33 Karki, R. & Kanneganti, T.-D. The ‘cytokine storm’: molecular mechanisms and therapeutic prospects. Trends in Immunology 42, 681-705 (2021).
34 Hotz, C. et al. Local delivery of mRNA-encoded cytokines promotes antitumor immunity and tumor eradication across multiple preclinical tumor models. Science Translational Medicine 13, eabc7804 (2021).
35 Pardi, N., Hogan, M. J., Porter, F. W. & Weissman, D. mRNA vaccines-a new era in vaccinology. Nature reviews Drug discovery 17, 261-279 (2018).
36 Huang, X. et al. The landscape of mRNA nanomedicine. Nature Medicine, 1-15 (2022).
37 Sahin, U., Kariko, K. & Türeci, Ö. mRNA-based therapeutics-developing a new class of drugs. Nature reviews Drug discovery 13, 759-780 (2014).
38 Hou, X., Zaks, T., Langer, R. & Dong, Y. Lipid nanoparticles for mRNA delivery. Nature Reviews Materials 6, 1078-1094 (2021).
39 Mendes, B. B. et al. Nanodelivery of nucleic acids. Nature Reviews Methods Primers 2, 1-21 (2022).
40 Baden, L. R. et al. Efficacy and safety of the mRNA-1273 SARS-COV-2 vaccine. New England journal of medicine (2020).
41 Skowronski, D. M. & De Serres, G. Safety and efficacy of the BNT162b2 mRNA Covid-19 vaccine. N Engl J Med 384, 1576-1577 (2021).
42 Oh, J. et al. Messenger RNA/polymeric carrier nanoparticles for delivery of heme oxygenase-1 gene in the post-ischemic brain. Biomaterials Science 8, 3063-3071 (2020).
43 Ho, W. et al. Next-Generation Vaccines: Nanoparticle-Mediated DNA and mRNA Delivery. Advanced Healthcare Materials 10, 2001812 (2021).
44 Li, F. et al. Co-delivery of oxygen and erlotinib by aptamer-modified liposomal complexes to reverse hypoxia-induced drug resistance in lung cancer. Biomaterials 145, 56-71 (2017).

What is claimed is:

1. A lipid nanoparticle comprising:
an ionizable cationic lipid compound comprising a reaction product of an amino alcohol and one or more lipid acids having from 4 to 26 carbons (C4-C26);
one or more other lipid components selected from the group consisting of a helper neutral lipid, a PEG-modified lipid, a phospholipid, cholesterol, and combinations thereof; and
a nucleic acid agent, the nucleic acid agent encoding an N-terminal domain of gasdermin.

2. The lipid nanoparticle of claim 1, wherein the nucleic acid agent comprises one or more of a DNA, an siRNA, a microRNA, an mRNA, a RNAi, an sgRNA, a plasmid, or antisense, single-stranded, double-stranded, circular, or self-amplifying varieties thereof.

3. The lipid nanoparticle of claim 2, wherein the nucleic acid agent comprises a single-agent mRNA.

4. The lipid nanoparticle of claim 3, wherein the single-agent mRNA comprises a circular mRNA or a self-amplifying mRNA.

5. The lipid nanoparticle of claim 1, wherein the gasdermin is selected from the group consisting of gasdermin-A (GSDM-A), gasdermin-B (GSDM-B), gasdermin-C(GSDM-C), gasdermin-D (GSDM-D), gasdermin-E (GSDM-E), and combinations thereof.

6. The lipid nanoparticle of claim 1, wherein the amino alcohol comprises two or more OH groups and the one or more lipid acids comprises octanoic acid (C8), decanoic acid (C10), dodecanoic acid (C12), tetradecanoic acid (C14), hexadecanoic acid (C16), octadecanoic acid (C18), oleic acid (C18:1), or linoleic acid (C18:2).

7. The lipid nanoparticle of claim 6, wherein the amino alcohol comprises 1,4-bis(2-hydroxyethyl) piperazine and the one or more lipid acids comprises linoleic acid (C18:2).

8. The lipid nanoparticle of claim 1, wherein the ionizable cationic lipid compound has a molecular weight in a range of from about 200 Daltons to about 2000 Daltons.

9. The lipid nanoparticle of claim 1, wherein the helper neutral lipid comprises one or more of 1,2-dioleoyl-sn-glycero-3-phosphoethanolamine (DOPE), 1,2-distearoyl-sn-glycero-3-phosphocholine (DSPC), and dioleoylphosphatidylcholine (DOPC).

10. The lipid nanoparticle of claim 1, wherein the PEG-modified lipid comprises 1,2-dimyristoyl-rac-glycero-3-methoxypolyethylene glycol (DMG-PEG).

11. The lipid nanoparticle of claim 1, wherein the ioniziable cationic lipid compound is synthesized by a one-step Candida antarctica Lipase-B (CALB) esterification.

12. The lipid nanoparticle of claim 1, wherein the ionizable cationic lipid compound comprises a structure of Formula (I) as follows:

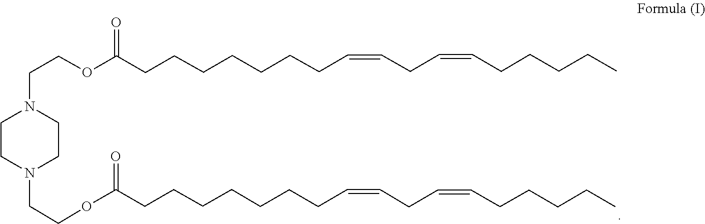 [SMILES: CCCCC/C=C\C/C=C\CCCCCCCC(=O)OCCN1CCN(CCOC(=O)CCCCCCC/C=C\C/C=C\CCCCC)CC1]

13. The lipid nanoparticle of claim 1, wherein the nucleic acid agent comprises a polynucleotide sequence having at least 90% sequence identity to SEQ ID NO. 1.

14. The lipid nanoparticle of claim 1, wherein the nucleic acid agent encodes a polypeptide sequence having at least 90% sequence identity to SEQ ID NO. 2.

15. A lipid nanoparticle comprising:
an ionizable cationic lipid compound comprising a structure of Formula (I) as follows: 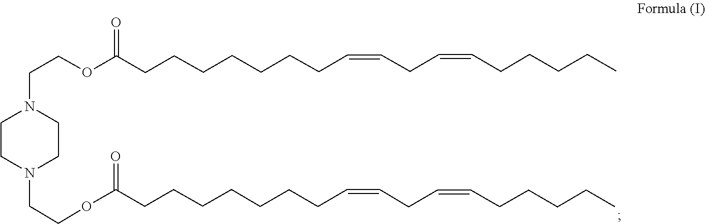 [SMILES: CCCCC/C=C\C/C=C\CCCCCCCC(=O)OCCN1CCN(CCOC(=O)CCCCCCC/C=C\C/C=C\CCCCC)CC1]
one or more other lipid components selected from the group consisting of a helper neutral lipid, a PEG-modified lipid, a phospholipid, cholesterol, and combinations thereof; and
a single-agent mRNA, the single-agent mRNA encoding an N-terminal domain of gasdermin and comprising a polynucleotide sequence having at least 90% sequence identity to SEQ ID NO. 1.

16. A method of treating a tumor, the method comprising administering a pharmaceutical composition to a subject in need thereof, the pharmaceutical composition comprising the lipid nanoparticle of claim 1.

17. The method of claim 16, wherein the pharmaceutical composition is administered intratumorally, subcutaneously, intramuscularly, intravenously, intraperitoneally, intradermally, or orally.

18. The method of claim 16, wherein the pharmaceutical composition is administered intratumorally.

19. The method of claim 16, wherein the tumor is resistant to cancer immunotherapy.

20. The method of claim 16, wherein the tumor is an anti-PD-1-resistant tumor.

21. The method of claim 16, wherein the treating the tumor induces a pyroptosis of cells in the tumor.

22. The method of claim 16, wherein the treating the tumor increases a concentration of pro-inflammatory cytokines in a serum or a tumor tissue of the subject, as measured by an enzyme-linked immunosorbent assay (ELISA).

23. The method of claim 22, wherein the pro-inflammatory cytokines are selected from the group consisting of tumor necrosis factor-alpha (TNF-α), interferon-gamma (IFN-γ), and combinations thereof.

24. The method of claim 16, wherein the subject is undergoing a cancer immunotherapy.

25. The method of claim 24, wherein the cancer immunotherapy comprises immune checkpoint blockade (ICB) therapy.

26. The method of claim 25, wherein the ICB therapy comprises administering an anti-PD-1 antibody to the subject.

27. The method of claim 25, wherein the ICB therapy is administered after administering the pharmaceutical composition comprising the lipid nanoparticle of claim 1.

28. The method of claim 27, wherein the ICB therapy is administered about 12 hours to about 2 days after administering the pharmaceutical composition comprising the lipid nanoparticle of claim 1.

29. The method of claim 28, wherein the ICB therapy is administered about 24 hours after administering the pharmaceutical composition comprising the lipid nanoparticle of claim 1.
